(19) United States
(12) Patent Application Publication
(10) Pub. No.: US 2023/0348398 A1
(43) Pub. Date: Nov. 2, 2023

(54) Benzimidazole derivatives

(71) Applicant: Hoffmann-La Roche Inc., Little Falls, NJ (US)

(72) Inventors: Guido GALLEY, Rheinfelden (DE); Katrin GROEBKE ZBINDEN, Liestal (CH); Daniel HUNZIKER, Moehlin (CH); Wolfgang GUBA, Muellheim (DE); Stefan BERCHTOLD, Binningen (CH); Danny KRUMM, Basel (CH); Daniela KRUMMENACHER, Zurich (CH); Manuel HILBERT, Village Neuf (FR)

(73) Assignee: Hoffmann-La Roche Inc., Little Falls, NJ (US)

(21) Appl. No.: 17/996,321

(22) PCT Filed: Apr. 14, 2021
(86) PCT No.: PCT/EP2021/059622

§ 371 Date: Oct. 14, 2022

(30) Foreign Application Priority Data
Apr. 16, 2020 (EP) 20169769.5

(51) Int. Cl. C07D 235/08; C07D 235/30; C07D 235/10; C07D 235/14; C07D 401/06; C07D 403/06

(52) U.S. Cl. CPC C07D 235/08; C07D 235/30; C07D 235/10; C07D 235/14; C07D 401/06; C07D 403/06

(57) ABSTRACT

The invention relates to a compound of formula (I) wherein R¹-R⁴ are as defined in the description and in the claims. The compound of formula (I) can be used as a medicament.

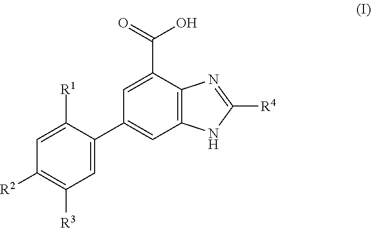

The present invention relates to organic compounds useful for therapy and/or prophylaxis in a mammal, and in particular to compounds that modulate cGAS activity.

In particular, the present invention relates to a compound of formula (I)

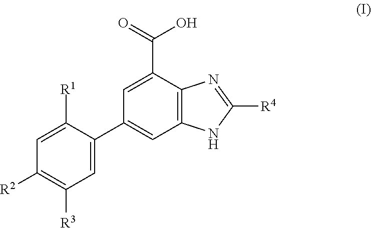

wherein
R¹ is alkyl or halogen;
R² is alkyl, halogen, haloalkyl, alkoxy or cycloalkyl;
R³ is hydrogen or halogen; and
R⁴ is hydrogen, (oxo-hexahydropyrrolo[1,2-a]pyrazinyl)alkyl, haloalkylpiperazinylalkyl, cycloalkylpiperazinylalkyl, phenylalkyl(oxopiperazinyl)alkyl, alkyl(oxopiperazinyl)alkyl, hydroxyalkyl, phenylamino, halopiperidinylalkyl, alkylcarbonylpiperazinylalkyl, phenyl(alkylamino)alkyl, phenyl alkylpiperazinylalkyl, phenylpiperazinylalkyl, oxopiperazinylalkyl, hydroxypiperidinylalkyl, alkylpiperazinylalkyl, dialkylaminoalkyl, piperidinylalkyl, phenylaminoalkyl, phenylalkyl, halophenylalkyl, morpholinylalkyl, haloalkyl, alkyl or phenylalkylamino;
or a pharmaceutically acceptable salt or ester thereof.

Cytokines are responsible for modulation of the innate immune response and the dysregulation of pro-inflammatory cytokines has been associated with severe systemic inflammation and autoimmune diseases, many of which lack efficient therapy as of today.

Vertebrates possess an innate and adaptive immune system as protection against pathogens and other challenges. The innate immune system is an evolutionary old system that is present beyond vertebrates. Unlike the adaptive immune system, it does not require priming or training, but works as a general physical barrier (e.g. skin) or by detection of specific patterns. One universal pattern to trigger the innate immune system is the detection of cytosolic double stranded DNA, which leads to Type I Interferon response. Sources of cytosolic dsDNA could be from bacterial or viral infection but as well accumulated self-DNA.

The cytosolic enzyme cyclic GMP-AMP Synthase (cGAS) is a sensor for cytosolic double stranded DNA. Binding of dsDNA results in the generation of the cyclic di-nucleotide 2,3-cGAMP by enzymatic linkage of ATP and GTP. 2,3-cGAMP acts as secondary messenger and binds to the Stimulator of Interferon Genes (STING), which resides in the endoplasmatic reticulum. Upon binding of 2,3-cGAMP, STING translocates to the perinuclear Golgi, where it associates with the TANK binding kinase 1 (TBK1) and recruits and phosphorylates Interferon Response Factor 3 (IRF3). Ultimately this results in the production of Type I Interferon (I IFN), other cytokines like IL-6, TNFα, IL1β and chemokines—essential factors for host defense against invading pathogens. However, inappropriate or chronic production of type I IFN and other pro-inflammatory cytokines are associated with severe systemic inflammation and autoimmune diseases. For instance, IFN signaling is involved in SLE, cutaneous skin diseases (dermatomyositis, and cutaneous lupus), interstitial pulmonary fibrosis, Sjogren syndrome, and type I diabetes (G. Trinchieri, J Exp Med. 2010 207(10): 2053-63). Other pro-inflammatory cytokine such as TNFα and IL1β play an important role in inflammatory bowel disease, NASH, juvenile inflammatory arthritis, ankylosing spondylitis and gout.

Chronic activation of cGAS/STING causes severe systemic inflammation. Evidence for its role in inflammation in the clinic comes from monogenic diseases. Patients with deficiencies in nucleic acid modifying enzymes, like Trex1, RNaseH2 and SAMHD1, suffer from Aicardi-Goutieres syndrome (AGS). The involvement of cGAS/STING was supported in Trex1 deficient mice that serve as a model for AGS.

Inhibition of the cGAS pathway which is upstream from the disease mediating cytokines is therefore a novel strategy in treating patients from multiple autoimmune diseases. Indications could include those linked to IFN signaling or those driven by TNFα and IL1β.

As of today many diseases caused by dysregulation of the innate immune system lack efficient therapies.

The compound of the invention binds to and modulates cGAS activity.

The compound of formula (I) is particularly useful in the treatment or prophylaxis of e.g. systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, sec.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, propyl, isopropyl, butyl, isobutyl, tert.-butyl, sec.-butyl and pentyl. Methyl and ethyl are particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl and cyclohexyl, cycloheptyl and cyclooctyl. A particular example of “cycloalkyl” is cyclopropyl.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. Particular examples of “alkoxy” are methoxy and ethoxy.

The term “oxy”, alone or in combination, signifies the —O— group.

The term “oxo”, alone or in combination, signifies the ═O group.

The terms “halogen” or “halo”, alone or in combination, signifies fluorine, chlorine, bromine or iodine and particularly fluorine, chlorine or bromine, more particularly fluorine and chlorine. The term “halo”, in combination with another group, denotes the substitution of said group with at least one halogen, particularly substituted with one to five halogens, particularly one to four halogens, i.e. one, two, three or four halogens.

The term “fluoro”, alone or in combination, signifies fluorine. The term “fluoro” in combination with another group, denotes the substitution of said group with at least one fluorine, particularly substituted with one to three fluorines, i.e. one, two or three fluorines.

The term “haloalkyl”, alone or in combination, denotes an alkyl group substituted with at least one halogen, particularly substituted with one to five halogens, particularly one to three halogens. Particular “haloalkyl” are fluoromethyl and fluoroethyl.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “alkylamino”, alone or in combination, denotes an amino group substituted with at least one alkyl, unless specified otherwise. Particular “aminoalkyl” are aminomethyl and aminoethyl. A particular “aminoalkyl” is aminomethyl”.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid, acetic acid, sodium and potassium.

The term “pharmaceutically acceptable esters” means that compounds of general formula (I) may be derivatised at functional groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compounds of general formula (I), similar to the metabolically labile esters, which are capable of producing the parent compounds of general formula (I) in vivo, are within the scope of this invention.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz). A particularly preferred protecting group is tert-butoxycarbonyl (Boc).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

The invention thus relates to:
A compound according to the invention wherein R¹ is alkyl or halogen;
A compound according to the invention wherein R² is alkyl, halogen, haloalkyl, alkoxy or cycloalkyl;
A compound according to the invention wherein R³ is hydrogen or halogen;
A compound according to the invention wherein R⁴ is hydrogen, (oxo-hexahydropyrrolo[1,2-a]pyrazinyl)alkyl, haloalkylpiperazinylalkyl, cycloalkylpiperazinylalkyl, phenylalkyl(oxopiperazinyl)alkyl, alkyl(oxopiperazinyl)alkyl, hydroxyalkyl, phenyl amino, halopiperidinylalkyl, alkylcarbonylpiperazinylalkyl, phenyl(alkylamino)alkyl, phenyl alkylpiperazinylalkyl, phenylpiperazinylalkyl, oxopiperazinylalkyl, hydroxypiperidinylalkyl, alkylpiperazinylalkyl, dialkylaminoalkyl, piperidinylalkyl, phenylaminoalkyl, phenylalkyl, halophenylalkyl, morpholinylalkyl, haloalkyl, alkyl or phenylalkylamino;
A compound according to the invention wherein R¹ is methyl or chlorine;
A compound according to the invention wherein R¹ is halogen;
A compound according to the invention wherein R¹ is chlorine;
A compound according to the invention wherein R² is methyl, chlorine, trifluoromethyl, ethoxy or cyclopropyl;
A compound according to the invention wherein R² is alkyl;
A compound according to the invention wherein R² is methyl;
A compound according to the invention wherein R³ is hydrogen or fluoride;
A compound according to the invention wherein R⁴ is hydrogen, (oxo-hexahydropyrrolo[1,2-a]pyrazinyl)methyl, trifluoroethylpiperazinylmethyl, cyclopropylpiperazinylmethyl, phenylmethyl(oxopiperazinyl)methyl, trifluoromethylpiperazinylmethyl, methyl(oxopiperazinyl)methyl, hydroxymethyl, phenylamino, difluoropiperidinylmethyl, methylcarbonylpiperazinylmethyl, phenyl(methylamino)methyl, phenylmethylpiperazinylmethyl, phenylpiperazinylmethyl, oxopiperazinylmethyl, hydroxypiperidinylmethyl, methylpiperazinylmethyl, diethylaminomethyl, piperidinylmethyl, phenylaminomethyl, phenylmethyl, chlorophenylmethyl, morpholinylmethyl, dimethylaminomethyl, trifluoromethyl, methyl or phenylmethylamino;
A compound according to the invention wherein R⁴ is hydrogen, (oxo-hexahydropyrrolo[1,2-a]pyrazinyl)alkyl, cycloalkylpiperazinylalkyl, hydroxyalkyl, halopiperidinylalkyl, phenylalkylpiperazinylalkyl, oxopiperazinylalkyl, alkylpiperazinylalkyl, piperidinylalkyl, phenyl aminoalkyl, phenylalkyl, morpholinylalkyl or phenylalkylamino; and
A compound according to the invention wherein R⁴ is hydrogen, (oxo-hexahydropyrrolo[1,2-a]pyrazinyl)methyl, cyclopropylpiperazinylmethyl, hydroxymethyl, halopiperidinylmethyl, phenylmethylpiperazinylmethyl, oxopiperazinylmethyl, methylpiperazinylmethyl, piperidinylmethyl, phenylaminomethyl, phenylmethyl, morpholinylmethyl or phenylmethylamino.

The invention further relates to a compound of formula (I) selected from
6-(2-chloro-4-cyclopropylphenyl)-1-benzimidazole-4-carboxylic acid;
6-(2-chloro-5-fluoro-4-methylphenyl)-1H-benzo[d]imidazole-4-carboxylic acid;
6-(2-chloro-4-methylphenyl)-1H-benzo[d]imidazole-4-carboxylic acid;
6-(2-chloro-4-ethoxyphenyl)-1H-benzo[d]imidazole-4-carboxylic acid;
6-(2-chloro-4-(trifluoromethyl)phenyl)-1H-benzo[d]imidazole-4-carboxylic acid;
6-(4-chloro-2-methylphenyl)-1H-benzo[d]imidazole-4-carboxylic acid;
6-(2-chloro-4-methylphenyl)-2-[(6-oxo-1,3,4,7,8,8-hexahydropyrrolo[1,2-a]pyrazin-2-yl)methyl]-1H-benzimidazole-4-carboxylic acid;
6-(2-chloro-4-methylphenyl)-2-[[4-(2,2,2-trifluoroethyl)piperazin-1-yl]methyl]-1H-benzimidazole-4-carboxylic acid;
6-(2-chloro-4-methylphenyl)-2-[(4-cyclopropylpiperazin-1-yl)methyl]-1H-benzimidazole-4-carboxylic acid;
2-[(4-benzyl-3-oxopiperazin-1-yl)methyl]-6-(2-chloro-4-methylphenyl)-1H-benzimidazole-4-carboxylic acid;
6-(2-chloro-4-methylphenyl)-2-[[3-(trifluoromethyl)piperazin-1-yl]methyl]-1H-benzimidazole-4-carboxylic acid;
6-(2-chloro-4-methylphenyl)-2-[(4-methyl-2-oxopiperazin-1-yl)methyl]-1H-benzimidazole-4-carboxylic acid;
6-(2-chloro-4-methylphenyl)-2-(hydroxymethyl)-1H-benzimidazole-4-carboxylic acid;
6-(2-chloro-4-methylphenyl)-2-(phenylamino)-1H-benzo[d]imidazole-4-carboxylic acid;
6-(2-chloro-4-methylphenyl)-2-[(4,4-difluoropiperidin-1-yl)methyl]-1H-benzimidazole-4-carboxylic acid;
2-[(4-acetylpiperazin-1-yl)methyl]-6-(2-chloro-4-methylphenyl)-1H-benzimidazole-4-carboxylic acid;
6-(2-chloro-4-methylphenyl)-2-[(N-methylanilino)methyl]-1H-benzimidazole-4-carboxylic acid;
2-[(4-benzylpiperazin-1-yl)methyl]-6-(2-chloro-4-methylphenyl)-1H-benzimidazole-4-carboxylic acid;
6-(2-chloro-4-methylphenyl)-2-[(4-phenylpiperazin-1-yl)methyl]-1H-benzimidazole-4-carboxylic acid;
6-(2-chloro-4-methylphenyl)-2-[(3-oxopiperazin-1-yl)methyl]-1H-benzimidazole-4-carboxylic acid;
6-(2-chloro-4-methylphenyl)-2-[(4-hydroxypiperidin-1-yl)methyl]-1H-benzimidazole-4-carboxylic acid;
6-(2-chloro-4-methylphenyl)-2-[(4-methylpiperazin-1-yl)methyl]-1-benzimidazole-4-carboxylic acid;
6-(2-chloro-4-methylphenyl)-2-(diethylaminomethyl)-1-benzimidazole-4-carboxylic acid;
6-(2-chloro-4-methylphenyl)-2-(piperidin-1-ylmethyl)-1H-benzimidazole-4-carboxylic acid;
2-(anilinomethyl)-6-(2-chloro-4-methylphenyl)-1H-benzimidazole-4-carboxylic acid;
2-benzyl-6-(2-chloro-4-methylphenyl)-1H-benzo[d]imidazole-4-carboxylic acid;
6-(2-chloro-4-methylphenyl)-2-[(4-chlorophenyl)methyl]-1H-benzimidazole-4-carboxylic acid;
6-(2-chloro-4-methylphenyl)-2-(morpholin-4-ylmethyl)-1H-benzimidazole-4-carboxylic acid;
6-(2-chloro-4-methylphenyl)-2-[(dimethylamino)methyl]-1H-benzimidazole-4-carboxylic acid;
6-(2-chloro-4-methylphenyl)-2-(trifluoromethyl)-1H-benzimidazole-4-carboxylic acid;
2-(benzylamino)-6-(2-chloro-4-methylphenyl)-1H-benzo[d]imidazole-4-carboxylic acid; and
6-(2-chloro-4-methylphenyl)-1H-benzo[d]imidazole-4-carboxylic acid;
   or a pharmaceutically acceptable salt or ester thereof.

The invention further relates in particular to a compound of formula (I) selected from
6-(2-chloro-5-fluoro-4-methylphenyl)-1H-benzo[d]imidazole-4-carboxylic acid;
6-(2-chloro-4-methylphenyl)-1H-benzo[d]imidazole-4-carboxylic acid;
6-(2-chloro-4-methylphenyl)-2-[(6-oxo-1,3,4,7,8,8a-hexahydropyrrolo[1,2-a]pyrazin-2-yl)methyl]-1H-benzimidazole-4-carboxylic acid;
6-(2-chloro-4-methylphenyl)-2-[(4-cyclopropylpiperazin-1-yl)methyl]-1H-benzimidazole-4-carboxylic acid;
6-(2-chloro-4-methylphenyl)-2-(hydroxymethyl)-1H-benzimidazole-4-carboxylic acid;
6-(2-chloro-4-methylphenyl)-2-[(4,4-difluoropiperidin-1-yl)methyl]-1H-benzimidazole-4-carboxylic acid;
2-[(4-benzylpiperazin-1-yl)methyl]-6-(2-chloro-4-methylphenyl)-1H-benzimidazole-4-carboxylic acid;
6-(2-chloro-4-methylphenyl)-2-[(3-oxopiperazin-1-yl)methyl]-1H-benzimidazole-4-carboxylic acid;
6-(2-chloro-4-methylphenyl)-2-[(4-methylpiperazin-1-yl)methyl]-1-benzimidazole-4-carboxylic acid;
6-(2-chloro-4-methylphenyl)-2-(piperidin-1-ylmethyl)-1H-benzimidazole-4-carboxylic acid;
2-(anilinomethyl)-6-(2-chloro-4-methylphenyl)-1H-benzimidazole-4-carboxylic acid;
2-benzyl-6-(2-chloro-4-methylphenyl)-1H-benzo[d]imidazole-4-carboxylic acid;
6-(2-chloro-4-methylphenyl)-2-(morpholin-4-ylmethyl)-1H-benzimidazole-4-carboxylic acid; and
2-(benzylamino)-6-(2-chloro-4-methylphenyl)-1H-benzo[d]imidazole-4-carboxylic acid;
   or a pharmaceutically acceptable salt or ester thereof.

The synthesis of the compound of formula (I) can, for example, be accomplished according to the following schemes.

The compound of formula (I) according to the invention, wherein R⁴ is hydrogen can be prepared according to scheme 1.

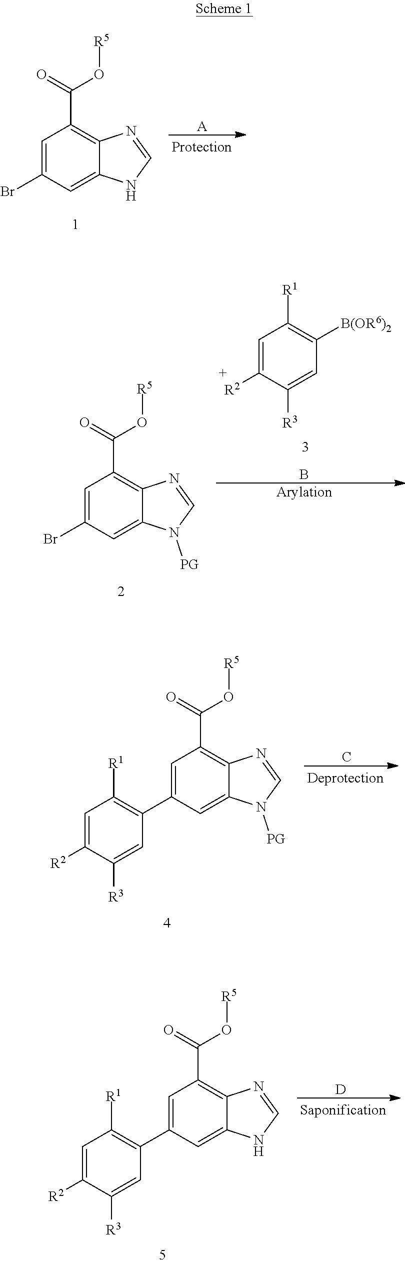

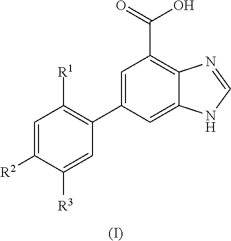

In scheme 1, R¹, R² and R³ are as defined above, R⁵ is alkyl; and R⁶ is hydrogen or alkyl. PG is protecting group.

In scheme 1, methyl is a convenient R⁵ and hydrogen is a convenient R⁶.

Step A: Protection of the nitrogen can be accomplished by reacting benzimidazole 1 with a suitable reagent such as di-tert-butyl-dicarbonate in presence of a base such as cesium carbonate, 4-dimethylaminopyridine (DMAP), triethylamine or ethyl diisopropylamine at 0° C.-75° C. in a suitable solvent such as dichloromethane, dichloroethane, tetrahydrofuran or acetonitrile.

A convenient protecting group is the tert-butyloxycarbonyl group and convenient conditions for the introduction are the use of di-tert-butyl-dicarbonate and DMAP in dichloromethane for 18 h at room temperature.

Step B: Coupling of the bromoderivative 1 with a suitable boronic acid or boronic acid ester 2 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of 1,1′-bis(diphenylphosphino)ferrocene-palladium(II)dichloride dichloromethane complex, X-phos and cesium carbonate in a mixture of dioxane and water at 100° C. for 1 h.

Step C: Deprotection can be accomplished by reaction of benzimidazole 4 with a suitable reagent such as trifluoroacetic acid or hydrochloric acid in dichloromethane or dioxane at room temperature for 1-18 h in case the convenient tert-butyloxycarbonyl group (PG=Boc) has been used. Furthermore the tert-butyloxycarbonyl group can be removed by heating with a base such as potassium carbonate or cesium carbonate in water and dioxane during the Suzuki reaction.

Convenient conditions are the heating with cesium carbonate in a mixture of water and dioxane at 90° C. for 1 h.

Step D: Saponification can be accomplished by reaction of the alkyl ester 5 with a base such as lithium hydroxide, sodium hydroxide, potassium hydroxide or the like in a suitable solvent such as water, tetrahydrofuran, ethanol, methanol or mixtures thereof for 1-18 h at 0° C. to 70° C. Saponification can be furthermore accomplished by reacting the alkyl ester 5 with an acid such as hydrobromic acid or hydrochlorid acid in water or acetic acid or a mixture thereof at 20° C.-110° C. for 1-24 h.

Advantageous conditions are the use of lithium hydroxide in a mixture of tetrahydrofuran and water at 65° C. for 18 h.

The compound of formula (I) according to the invention, wherein R⁴ is not hydrogen, phenylamino or phenylaminoalkyl, can be prepared according to scheme 2.

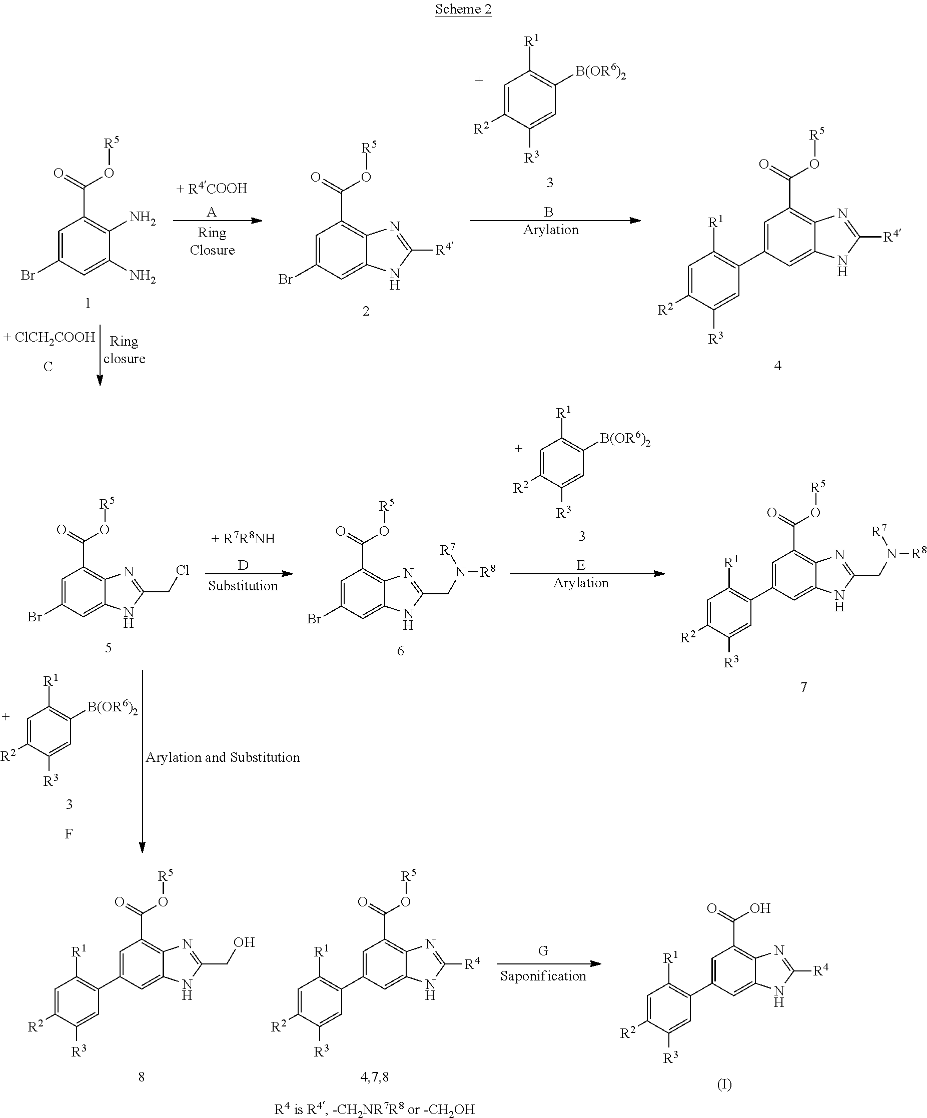

In scheme 2, R¹-R⁴ are as defined above; R^{4′} is phenylalkyl, halophenylalkyl, haloalkyl or alkyl; R⁵ is alkyl; R⁶ is hydrogen or alkyl; R⁷ and R⁸ are independently selected from hydrogen, alkyl and phenyl, or R⁷ and R⁸, together with the nitrogen atom to which they are attached, form (oxo-hexahydropyrrolo[1,2-a]pyrazinyl), haloalkylpiperazinyl, cycloalkylpiperazinyl, phenylalkyl(oxopiperazinyl), alkyl(oxopiperazinyl), halopiperidinyl, alkylcarbonylpiperazinyl, phenylalkylpiperazinyl, phenylpiperazinyl, oxopiperazinyl, hydroxypiperidinyl, alkylpiperazinyl, piperidinyl or morpholinyl.

Conveniently in scheme 2, R⁶ is hydrogen.

Step A: Ring closure to form benzimidazole 2 can be accomplished by reacting diamine 1 with an acid R^{4′}COOH with or without addition of an inorganic acid chloride such as phosphorous oxychloride or thionyl chloride with or without using an additional solvent such as 1,2-dichloroethane, dichloromethane or toluene at 0° C.-120° C. for 1 h-18 h.

Convenient conditions are the use of phosphorous oxychloride without additional solvent at 100° C. for 2 h.

Step B: Coupling of the bromoderivative 2 with a suitable boronic acid or boronic acid ester 3 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of tris(dibenzylideneacetone)dipalladium chloroform adduct, X-phos and potassium carbonate in a mixture of dioxane and water at 100° C. for 1 h.

Step C: Ring closure to form benzimidazole 5 can be accomplished by reacting diamine 1 with an chloroacetic acid derivative in an organic solvent or in water. Chloroacetic acid can be used in presence of hydrochloric acid in water at elevated temperatures followed by re-esterification with methanol and sulfuric acid.

Convenient conditions are the use of chloroacetic acid in aqueous hydrochloric acid at 100° C. for 18 h followed by reaction with methanol and concentrated sulfuric acid at reflux for 18 h.

Step D: Substitution to form benzimidazole 6 can be accomplished by reacting with the amine R⁷R⁸NH in an appropriate solvent such as tetrahydrofuran, dichloromethane, dioxane, dimethylformamide or the like with or without presence of a base such as triethylamine, ethyldiisopropylamine or N-methylmorpholine at 0° C.-140° C. for 1-24 h.

Convenient conditions are the use of triethylamine and tetrahydrofuran at room temperature for 18 h.

Step E: Coupling of the bromoderivative 6 with a suitable boronic acid or boronic acid ester 3 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of tris(dibenzylideneacetone)dipalladium chloroform adduct, X-phos and potassium carbonate in a mixture of dioxane and water at 100° C. for 1 h.

Step F: Coupling of the bromoderivative 5 with a suitable boronic acid or boronic acid ester 3 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs.

Convenient conditions are the use of tris(dibenzylideneacetone)dipalladium chloroform adduct, X-phos and potassium carbonate in a mixture of dioxane and water at 100° C. for 1 h.

Step G: Saponification can be accomplished by reaction of the alkyl ester of any one of compounds 4, 7 or 8 with a base such as lithium hydroxide, sodium hydroxide, potassium hydroxide or the like in a suitable solvent such as water, tetrahydrofuran, ethanol, methanol or mixtures thereof for 1-18 h at 0° C. to 70° C. Saponification can be furthermore accomplished by reacting the alkyl ester of any one of compounds 4, 7 or 8 with an acid such as hydrobromic acid or hydrochlorid acid in water or acetic acid or a mixture thereof at 20° C.-110° C. for 1-24 h.

Advantageous conditions are the use of lithium hydroxide in a mixture of tetrahydrofuran and water at 65° C. for 4 h.

The compound of formula (I) according to the invention, wherein R⁴ is phenylamino or phenylaminoalkyl, can be prepared according to scheme 3.

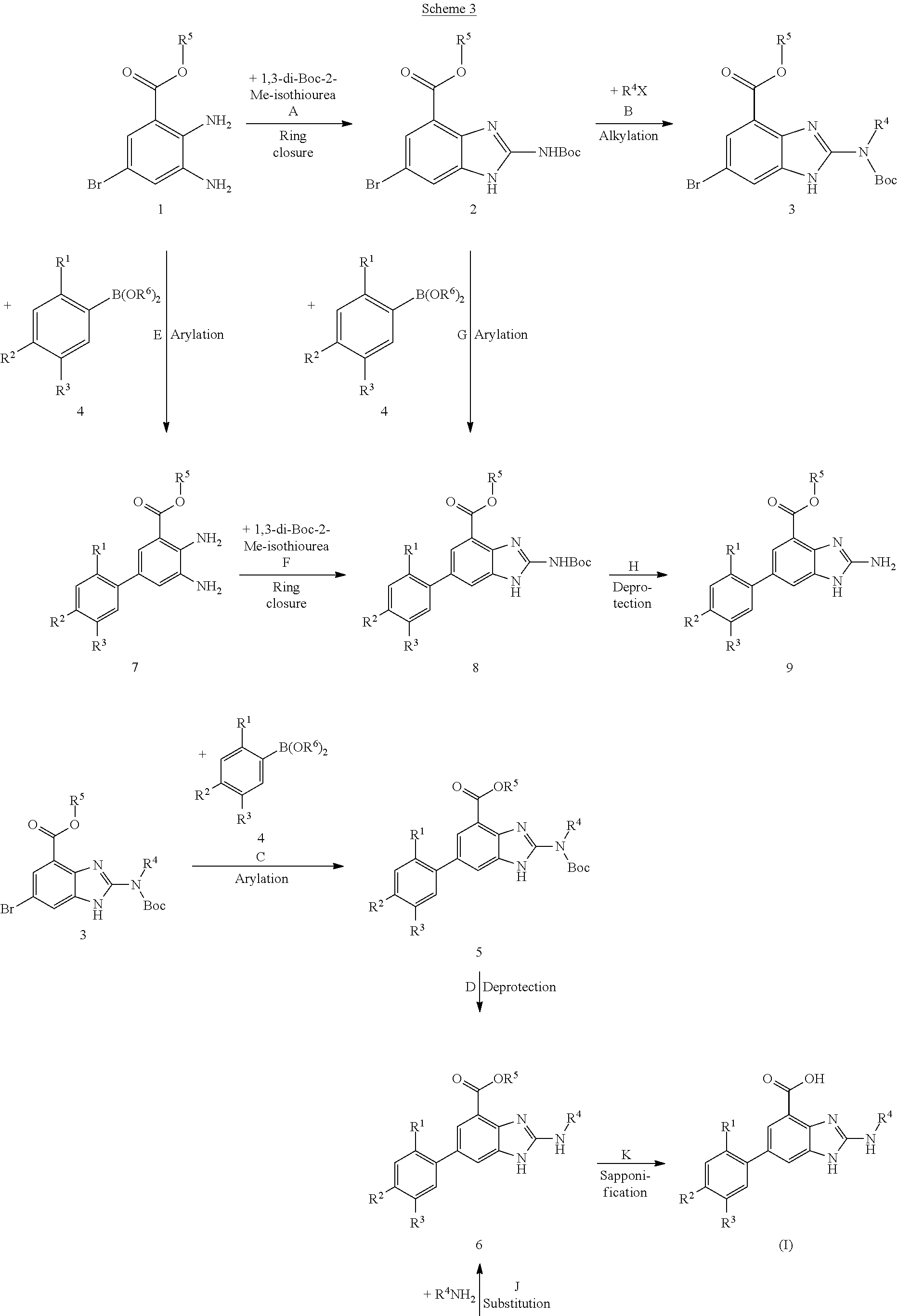

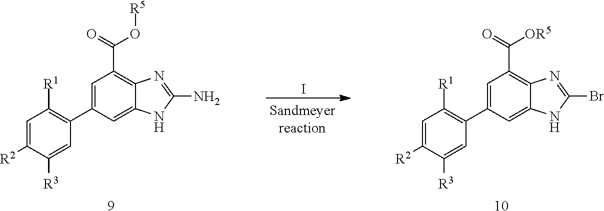

In scheme 3, R¹-R³ are as defined above; R⁵ is alkyl; R⁶ is hydrogen or alkyl; X is a leaving group, such as a halogen, mesylate or tosylate.

Conveniently in scheme 3, R⁵ is methyl.

Conveniently in scheme 3, R⁶ is hydrogen.

Step A: Formation of benzimidazole derivative 2 can be accomplished by reaction of diamino compound 1 with 1,3-di-boc-2-methyl-isothiourea and an acid such as and (+)-camphor-10-sulfonic acid monohydrate in ethanol or methanol at 25° C.-75° C. for 1 h-24 h.

Convenient conditions are the use of 1,3-di-boc-2-methyl-isothiourea and (+)-camphor-10-sulfonic acid monohydrate in ethanol for 3 h at reflux.

Step B: Alkylation/benzylation can be accomplished by reacting the benzimidazole 2 with R⁴—X, wherein R⁴ is an optionally substituted alkyl or benzyl and wherein X is a leaving group. Examples for R⁴—X are alkylchlorides, alkylbromides, alkyliodides, alkyltosylates, benzylbromides, benzylchlorides or the like and a base such as cesium carbonate, potassium carbonate, sodium carbonate, triethylamine or ethyldiisopropylamine in a solvent such as dioxane, dimethylacetamide, dimethylformamide, tetrahydrofuran at 0° C.-150° C. for 1 h to 18 h. If regioisomeric mixtures of alkylation products are obtained they can be separated by column chromatography on silica gel using mixtures of organic solvents such as heptane, ethylacetate, methanol and dichloromethane to yield the described regioisomer as a pure compound.

Convenient conditions are the use of benzylbromide and cesium carbonate in dimethylformamide for 18 h at room temperature.

Step C: Coupling of the bromoderivative 3 with a suitable boronic acid or boronic acid ester 4 and subsequent removal of the Boc group can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate in a suitable solvent such as dioxane or tetrahydrofuran in presence of water at 50° C. to 180° C. for 5 min to 18 hrs with or without microwave irradiation.

Convenient conditions are the use of tris(dibenzylideneacetone)dipalladium chloroform adduct, X-phos and potassium carbonate in a mixture of dioxane and water at 100° C. for 1 h.

Step D: Deprotection can be accomplished by reaction of benzimidazole 5 with a suitable reagent such as trifluoroacetic acid or hydrochloric acid in dichloromethane or dioxane at room temperature for 1-18 hours.

Convenient conditions are the use of hydrochloric acid in dioxane at 20° C. for 1-2 days.

Step E: Coupling of the bromoderivative 1 with a suitable boronic acid or boronic acid ester 4 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of tris(dibenzylideneacetone)dipalladium chloroform adduct, X-phos and potassium carbonate in a mixture of dioxane and water at 100° C. for 1 h.

Step F: Formation of benzimidazole derivative 8 can be accomplished by reaction of diamino compound 7 with 1,3-di-boc-2-methyl-isothiourea and an acid such as (+)-camphor-10-sulfonic acid monohydrate in ethanol or methanol at 25° C.-75° C. for 1 h-24 h.

Convenient conditions are the use of 1,3-di-boc-2-methyl-isothiourea and (+)-camphor-10-sulfonic acid monohydrate in ethanol for 3 h at reflux.

Step G: Coupling of the bromoderivative 2 with a suitable boronic acid or boronic acid ester 4 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of tris(dibenzylideneacetone)dipalladium chloroform adduct, X-phos and potassium carbonate in a mixture of dioxane and water at 100° C. for 1 h.

Step H: Deprotection can be accomplished by reaction of benzimidazole 8 with a suitable reagent such as trifluoroacetic acid or hydrochloric acid in dichloromethane or dioxane at room temperature for 1-18 hours.

Convenient conditions are the use of hydrochloric acid in dioxane at 20° C. for 1-2 days.

Step I: Formation of bromo compound 10 can be accomplished by reaction of benzimidazole compound 9 with sodium nitrite an acid such as hydrochloric acid in water, or by reaction with tert.-butyl nitrite or isoamyl nitrite in an organic solvent such as acetonitrile at 25-80° C.

Convenient conditions are the use of tert.-butyl nitrite and copper (II) bromide in acetonitrile at 75° C. for 2 hours.

Step J: Substitution can be accomplished by reaction of benzimidazole compound 10 with an amine R⁴—NH₂ with or without an additional base and with or without an organic solvent such as dimethylformamide, dimethylacetamide, tetrahydrofuran, dichloromethane or 1,2-dichloroethane.

Convenient conditions are the use of an excess of the amine and heating the mixture to 90° C. for 18 h.

Step K: Saponification can be accomplished by reaction of the alkyl ester 6 with a base such as lithium hydroxide, sodium hydroxide, potassium hydroxide or the like in a suitable solvent such as water, tetrahydrofuran, ethanol, methanol or mixtures thereof for 1-18 h at 0° C. to 70° C. Saponification can be furthermore accomplished by reacting the alkyl ester 5 with an acid such as hydrobromic acid or hydrochlorid acid in water or acetic acid or a mixture thereof at 20° C.-110° C. for 1-24 h.

Advantageous conditions are the use of lithium hydroxide in a mixture of tetrahydrofuran and water at around 65° C. for 4 h.

The invention thus also relates to a process for the preparation of a compound according to the invention, comprising the saponification of a compound of formula (A1)

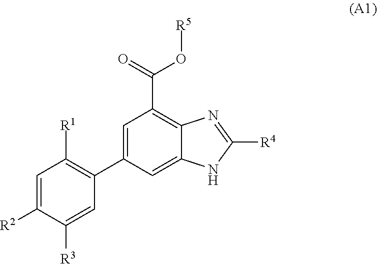

in the presence of a base or an acid;
wherein R¹, R², R³ and R⁴ are as defined above and R⁵ is alkyl.
R⁵ is conveniently methyl.

The saponification can be conveniently carried out in a solvent. The solvent can be for example water, tetrahydrofuran, ethanol, methanol, acetic acid or mixtures thereof.

In the saponification the base can be for example lithium hydroxide, sodium hydroxide or potassium hydroxide.

In the saponification the acid can be for example hydrobromic acid or hydrochlorid acid.

Convenient conditions for the saponification under basic conditions can be between around 0° C.-100° C., particularly around 40° C.-90° C., more particularly around 50° C.-80° C., in particular around 65° C.

Preferred conditions for saponification under basic conditions are the use of lithium hydroxide in a mixture of tetrahydrofuran and water at around 65° C. for around 4 h.

Convenient conditions for the saponification under acidic conditions can be between around 0° C.-170° C., particularly around 20° C.-150° C., more particularly around 80° C.-120° C., in particular around 110° C.

Preferred conditions for saponification under acidic conditions are the use of hydrobromic acid in acetic acid at around 110° C. for around 3 h.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention also relates in particular to:
A compound of formula (I) for use in the treatment of a disease modulated by cGAS;
The use of a compound of formula (I) for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);
The use of a compound of formula (I) for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);
A compound of formula (I) for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); and
A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or

Aicardi-Goutieres syndrome (AGS), which method comprises administering an effective amount of a compound of formula (I) to a patient in need thereof.

The invention will now be illustrated by the following examples which have no limiting character.

### EXAMPLES

### Abbreviations

DCM=dichloromethane; DMSO=dimethyl sulfoxide; ESI=electrospray ionization; EtOAc=ethyl acetate; HPLC=high performance liquid chromatography; MeOH=methanol; MS=mass spectrometry; RT=room temperature; TFA=trifluoroacetic acid; THF=tetrahydrofuran.

### Example 1

### 6-(2-Chloro-4-cyclopropylphenyl)-1-benzimidazole-4-carboxylic acid

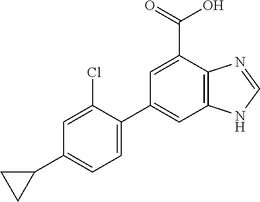 [SMILES: O=C(O)c1cc(-c2ccc(C3CC3)cc2Cl)cc2[nH]cnc12]

### a) 1-(tert-Butyl) 4-methyl 6-bromo-1H-benzo[d]imidazole-1,4-dicarboxylate

To a suspension of methyl 6-bromo-1H-benzo[d]imidazole-4-carboxylate (750 mg, 2.94 mmol, Eq: 1) and N-ethyldiisopropylamine (988 mg, 1.31 ml, 7.64 mmol, Eq: 2.6) in dichloromethane (20 ml) were added di-tert-butyl dicarbonate (1.35 g, 6.17 mmol, Eq: 2.1) and DMAP (36.7 mg, 294 μmol, Eq: 0.1). The reaction was stirred at room temperature overnight, then it was poured into 50 ml of water and extracted with EtOAc (2×50 mL). The organic layers were dried over Na₂SO₄ and concentrated in vacuo. The crude material was purified by flash chromatography (silica gel, 40 g, 0% to 100% EtOAc in heptane) to obtain the title compound 1-(tert-butyl) 4-methyl 6-bromo-1H-benzo[d]imidazole-1,4-dicarboxylate (846.9 mg, 2.38 mmol, 80.8% yield) as white solid, MS (ESI): 355.029 [M+H]+.

### b) Methyl 6-(2-chloro-4-cyclopropylphenyl)-1H-benzo[d]imidazole-4-carboxylate

To a light yellow solution of 1-(tert-butyl) 4-methyl 6-bromo-1H-benzo[d]imidazole-1,4-dicarboxylate (50 mg, 134 μmol, Eq: 1) in 1,4-dioxane (1.2 ml) was added (2-chloro-4-cyclopropylphenyl)boronic acid (39.4 mg, 201 Eq: 1.5). Cesium carbonate (88 mg, 267 μmol, Eq: 2) dissolved in water (0.6 ml) was added. The reaction mixture was degassed with Argon before 1,1′-bis(diphenylphosphino)ferrocene-palladium(II)dichloride dichloromethane complex (5.46 mg, 6.69 μmol, Eq: 0.05) was added. The mixture was heated to 90° C. for 1 hour. The reaction mixture was poured into 20 ml of water and extracted with EtOAc (3×20 ml). The crude material was purified by flash chromatography (silica gel, 12 g, 0% to 10% MeOH in DCM) to obtain the title compound methyl 6-(2-chloro-4-cyclopropylphenyl)-1H-benzo[d]imidazole-4-carboxylate (31.6 mg, 95.4 μmol, 71.4% yield) as light brown solid, MS (ESI): 327.13 [M+H]+.

### c) 6-(2-Chloro-4-cyclopropylphenyl)-1H-benzo[d]imidazole-4-carboxylic acid

To a solution of methyl 6-(2-chloro-4-cyclopropylphenyl)-1H-benzo[d]imidazole-4-carboxylate (30 mg, 91.8 μmol, Eq: 1) in tetrahydrofuran (1.2 ml) was added lithium hydroxide monohydrate (7.7 mg, 184 Eq: 2) dissolved in water (0.6 ml). The reaction mixture was warmed to 65° C. and stirred for 3 h. For work-up HCl (2M, 91.8 μl, 184 μmol, Eq: 2) was added, the mixture was concentrated in vacuo. The residue was treated with methyltetrahydrofuran and the crystals were filtered off to obtain the title compound 6-(2-chloro-4-cyclopropylphenyl)-1-benzimidazole-4-carboxylic acid (20.6 mg, 65 μmol, 70.8% yield) as white solid, MS (ESI): 313.074 [M+H]+.

### Example 2

### 6-(2-Chloro-5-fluoro-4-methylphenyl)-1H-benzo[d]imidazole-4-carboxylic acid

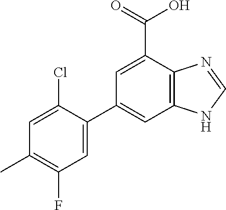 [SMILES: Cc1cc(Cl)c(-c2cc(C(=O)O)c3nc[nH]c3c2)cc1F]

The title compound was obtained in comparable yield analogous to the procedure described for Example 1 using (2-chloro-5-fluoro-4-methylphenyl)boronic acid instead of (2-chloro-4-cyclopropylphenyl)boronic acid in step b), off-white solid, MS (ESI): 305.1 [M+H]+.

### Example 3

### 6-(2-Chloro-4-methylphenyl)-1H-benzo[d]imidazole-4-carboxylic acid

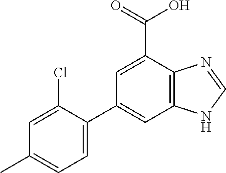 [SMILES: Cc1ccc(-c2cc(C(=O)O)c3nc[nH]c3c2)c(Cl)c1]

The title compound was obtained in comparable yield analogous to the procedure described for Example 1 using (2-chloro-4-methylphenyl)boronic acid instead of (2-chloro-5-fluoro-4-methylphenyl)boronic acid in step b), off-white solid, MS (ESI): 287.1 [M+H]+.

### Example 4

### 6-(2-Chloro-4-ethoxyphenyl)-1H-benzo[d]imidazole-4-carboxylic acid

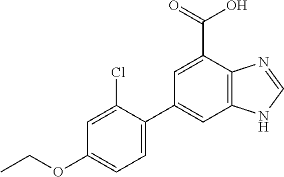 [SMILES: CCOc1ccc(-c2cc(C(=O)O)c3nc[nH]c3c2)c(Cl)c1]

### a) Methyl 6-(2-chloro-4-ethoxyphenyl)-1H-benzo[d]imidazole-4-carboxylate

(2-Chloro-4-ethoxyphenyl)boronic acid (84.6 mg, 401 μmol, Eq: 1.5) and 1-(tert-butyl) 4-methyl 6-bromo-1H-benzo[d]imidazole-1,4-dicarboxylate (100 mg, 267 μmol, Eq: 1) were solved in 1,4-dioxane (6 ml) and water (3 ml). Cesium carbonate (352 mg, 1.07 mmol, Eq: 4) was added and the mixture was degassed by bubbling argon through the mixture (5 min) 1,1′-bis(diphenylphosphino)ferrocene-palladium(II)dichloride dichloromethane complex (10.9 mg, 13.4 μmol, Eq: 0.05) was added. The reaction was stirred in a sealed tube at 90° C. The mixture was taken up in EtOAc and washed with water, saturated NH₄Cl solution and brine. The organic layer was dried over Na₂SO₄, filtrated and evaporated. The residue was diluted with CH₂Cl₂, evaporated with silica gel to dryness and purified by flash chromatography (silica gel, 40 g, with 0% to 100% EtOAc in heptane) to obtain the title compound methyl 6-(2-chloro-4-ethoxyphenyl)-1H-benzo[d]imidazole-4-carboxylate (58.6 mg, 177 μmol, 44.1% yield) as off-white solid, MS (ESI): 331.2 [M+H]+.

### b) 6-(2-Chloro-4-ethoxyphenyl)-1H-benzo[d]imidazole-4-carboxylic acid

Methyl 6-(2-chloro-4-ethoxyphenyl)-1H-benzo[d]imidazole-4-carboxylate (56 mg, 169 μmol, Eq: 1) was solved in acetic acid (1.18 g, 1.09 ml, 19.6 mmol, Eq: 116). Hydrobromic acid (816 mg, 544 μl, 4.84 mmol, Eq: 28.6) was added at room temperature. The mixture was stirred at 110° C. for 3 h, then it was concentrated at high vacuum at 50° C. The residue was taken up in 15% Na₂CO₃-solution, the aqueous layer was extracted twice with diethylether, the organic layer was washed with water and the combined aqueous layers were acidified with HCl 37% to pH3 and extracted twice with 2-methyltetrahydrofuran. The combined organic layers were dried over Na₂SO₄, filtrated and evaporated. The crude product was purified by preparative HPLC (Gemini NX, 12 nm, 5 μm, 100×30 mm, eluent acetonitrile/water). The solvent acetonitrile was evaporated and the remaining aqueous layer was extracted with 2-methyltetrahydrofuran, the organic layer was washed with water, then dried over Na₂SO₄, filtrated and evaporated to yield the title compound 6-(2-chloro-4-ethoxyphenyl)-1H-benzo[d]imidazole-4-carboxylic acid (16.6 mg, 52.4 μmol, 31.0% yield) as light yellow solid, MS (ESI): 317.1 [M+H]+.

### Example 5

### 6-(2-Chloro-4-(trifluoromethyl)phenyl)-1H-benzo[d]imidazole-4-carboxylic acid

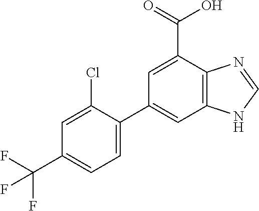 [SMILES: O=C(O)c1cc(-c2ccc(C(F)(F)F)cc2Cl)cc2[nH]cnc12]

The title compound was obtained in comparable yield analogous to the procedure described for Example 4 using (2-chloro-4-(trifluoromethyl)phenyl)boronic acid instead of (2-chloro-4-ethoxyphenyl)boronic acid in step a), light yellow solid, MS (ESI): 341.1 [M+H]+.

### Example 6

### 6-(4-Chloro-2-methylphenyl)-1H-benzo[d]imidazole-4-carboxylic acid

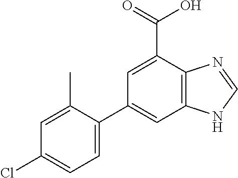 [SMILES: Cc1cc(Cl)ccc1-c1cc(C(=O)O)c2nc[nH]c2c1]

The title compound was obtained in comparable yield analogous to the procedure described for Example 4 using (4-chloro-2-methylphenyl)boronic acid instead of (2-chloro-4-ethoxyphenyl)boronic acid in step a), light yellow solid, MS (ESI): 287.1 [M+H]+.

### Example 7

### 6-(2-Chloro-4-methylphenyl)-2-[(6-oxo-1,3,4,7,8,8a-hexahydropyrrolo[1,2-a]pyrazin-2-yl)methyl]-1H-benzimidazole-4-carboxylic acid

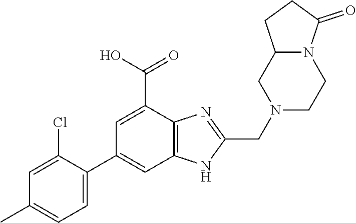 [SMILES: Cc1ccc(-c2cc(C(=O)O)c3nc(CN4CCN5C(=O)CCC5C4)[nH]c3c2)c(Cl)c1]

### a) Methyl 6-bromo-2-(chloromethyl)-1H-benzo[d]imidazole-4-carboxylate

To a light yellow solution of methyl 2,3-diamino-5-bromobenzoate (10 g, 40.8 mmol, Eq: 1) in water (100 ml) and HCl cone (100 ml) was added chloroacetic acid (4.24 g, 44.9 mmol, Eq: 1.1). The mixture was heated to 100° C. overnight. The reaction mixture was poured into 25 ml of water and extracted with EtOAc (3×100 ml). The organic layers were dried over MgSO₄ and concentrated in vacuo to obtain the crude intermediate 6-bromo-2-(chloromethyl)-1H-benzo[d]imidazole-4-carboxylic acid (7.14 g, 24.7 mmol, 60.4% yield) as red solid, which was taken up with methanol (150 ml). Sulfuric acid (16 g, 8.7 ml, 163 mmol, Eq: 4) was added. The mixture was heated to reflux over night. The crude reaction mixture was partly concentrated in vacuo, then it was slowly poured into 150 ml saturated NaHCO₃ solution and extracted with EtOAc (3×150 ml). The organic layers were dried over MgSO₄ and concentrated in vacuo. The crude material was purified by flash chromatography (silica gel, 80 g, 0% to 100% EtOAc in heptane) to afford the title compound methyl 6-bromo-2-(chloromethyl)-1H-benzo[d]imidazole-4-carboxylate (4.72 g, 15.3 mmol, 37.5% yield) as brown semisolid, (ESI): 304.94 [M+H]+.

### b) Methyl 6-(2-chloro-4-methylphenyl)-2-((6-oxohexahydropyrrolo[1,2-a]pyrazin-2(1H)-yl)methyl)-1H-benzo[d]imidazole-4-carboxylate

To a light brown solution of methyl 6-bromo-2-(chloromethyl)-1H-benzo[d]imidazole-4-carboxylate (100 mg, 329 μmol, Eq: 1) and triethylamine (133 mg, 184 μl, 1.32 mmol, Eq: 4) in tetrahydrofuran (2 ml) was added hexahydropyrrolo[1,2-a]pyrazin-6(2H)-one (55.4 mg, 395 μmol, Eq: 1.2). The reaction mixture was stirred at room temp overnight. The mixture was poured into 10 ml of water and extracted twice with EtOAc (2×10 ml). The organic layers were dried over MgSO₄ and concentrated in vacuo to obtain the intermediate which was taken up in dioxane (1.5 ml). (2-Chloro-4-methylphenyl)boronic acid (56.1 mg, 329 μmol, Eq: 1) was added. Potassium phosphate (210 mg, 81.8 μl, 988 μmol, Eq: 3) dissolved in water (0.375 ml) was added. The mixture was degassed during 2 min before X-phos (7.85 mg, 16.5 μmol, Eq: 0.05) and tris(dibenzylideneacetone)dipalladium chloroform adduct (8.53 mg, 8.24 μmol, Eq: 0.025) were added. The mixture was heated to 100° C. for 1 hours. The reaction mixture was poured into 20 ml of water and extracted with EtOAc (3×20 ml). The organic layers were dried over MgSO₄ and concentrated in vacuo. The crude material was purified by flash chromatography (silica gel, 12 g, 0% to 5% MeOH in DCM) to afford the title compound methyl 6-(2-chloro-4-methylphenyl)-2-((6-oxohexahydropyrrolo[1,2-a]pyrazin-2(1H)-yl)methyl)-1H-benzo[d]imidazole-4-carboxylate (22.5 mg, 41.8 μmol, 12.7% yield) as light brown solid, MS (ESI): 453.3 [M+H]+.

### c) 6-(2-chloro-4-methylphenyl)-2-[(6-oxo-1,3,4,7,8,8a-hexahydropyrrolo[1,2-a]pyrazin-2-yl)methyl]-1H-benzimidazole-4-carboxylic acid

To a light yellow solution of methyl 6-(2-chloro-4-methylphenyl)-24(6-oxohexahydropyrrolo[1,2-a]pyrazin-2(1H)-yl)methyl)-1H-benzo[d]imidazole-4-carboxylate (20 mg, 44.2 μmol, Eq: 1) in tetrahydrofuran (1.5 ml) was added lithium hydroxide monohydrate (3.71 mg, 88.3 Eq: 2) dissolved in water (375 μl). The reaction mixture was heated to 65° C. and stirred during 4 hours. The mixture was quenched with HCl (2M, 44.2 μl, 88.3 μmol, Eq: 2) and concentrated in vacuo. The crude material was purified by preparative HPLC (Gemini NX, 12 nm, 5 μm, 100×30 mm, eluent acetonitrile/water) to obtain the title compound 6-(2-chloro-4-methylphenyl)-2-[(6-oxo-1,3,4,7,8,8a-hexahydropyrrolo[1,2-a]pyrazin-2-yl)methyl]-1H-benzimidazole-4-carboxylic acid (7 mg, 15.2 μmol, 34.4% yield) as white solid, MS (ESI): 439.27 [M+H]+.

### Example 8

### 6-(2-Chloro-4-methylphenyl)-2-((4-(2,2,2-trifluoroethyl)piperazin-1-yl)methyl)-1H-benzo[d]imidazole-4-carboxylic acid

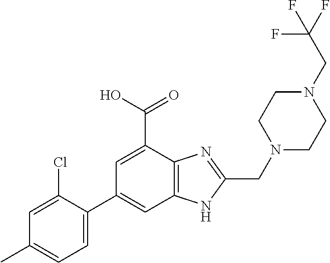 [SMILES: Cc1ccc(-c2cc(C(=O)O)c3nc(CN4CCN(CC(F)(F)F)CC4)[nH]c3c2)c(Cl)c1]

The title compound was obtained in comparable yield analogous to the procedure described for Example 7 using 1-(2,2,2-trifluoroethyl)piperazine hydrochloride instead of hexahydropyrrolo[1,2-a]pyrazin-6(2H)-one in step b), light yellow solid, (MS (ESI): 467.33 [M+H]+.

### Example 9

### 6-(2-Chloro-4-methylphenyl)-2-[(4-cyclopropylpiperazin-1-yl)methyl]-1H-benzimidazole-4-carboxylic acid

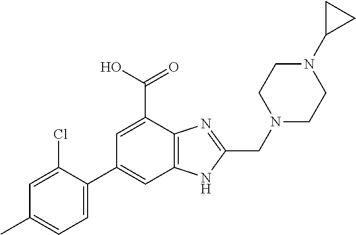 [SMILES: Cc1ccc(-c2cc(C(=O)O)c3nc(CN4CCN(C5CC5)CC4)[nH]c3c2)c(Cl)c1]

The title compound was obtained in comparable yield analogous to the procedure described for Example 7 using 1-cyclopropylpiperazine instead of hexahydropyrrolo[1,2-a]pyrazin-6(2H)-one in step b), light brown solid, (MS (ESI): 425.31 [M+H]+.

### Example 10

### 2-[(4-Benzyl-3-oxopiperazin-1-yl)methyl]-6-(2-chloro-4-methylphenyl)-1H-benzimidazole-4-carboxylic acid

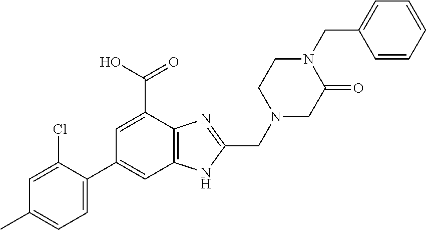 [SMILES: Cc1ccc(-c2cc(C(=O)O)c3nc(CN4CCN(Cc5ccccc5)C(=O)C4)[nH]c3c2)c(Cl)c1]

The title compound was obtained in comparable yield analogous to the procedure described for Example 7 using 1-benzylpiperazin-2-one instead of hexahydropyrrolo[1,2-a]pyrazin-6(2H)-one in step b), light yellow solid, (MS (ESI): 489.31 [M+H]+.

### Example 11

### 6-(2-Chloro-4-methylphenyl)-2-[[3-(trifluoromethyl)piperazin-1-yl]methyl]-1H-benzimidazole-4-carboxylic acid

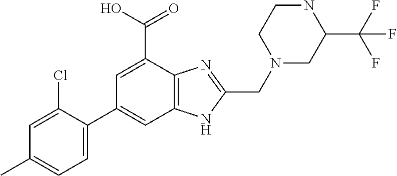 [SMILES: Cc1ccc(-c2cc(C(=O)O)c3nc(CN4CCNC(C(F)(F)F)C4)[nH]c3c2)c(Cl)c1]

The title compound was obtained in comparable yield analogous to the procedure described for Example 7 using 2-(trifluoromethyl)piperazine instead of hexahydropyrrolo[1,2-a]pyrazin-6(2H)-one in step b), light yellow solid, MS (ESI): 453.22 [M+H]+.

### Example 12

### 6-(2-Chloro-4-methylphenyl)-2-[(4-methyl-2-oxopiperazin-1-yl)methyl]-1H-benzimidazole-4-carboxylic acid

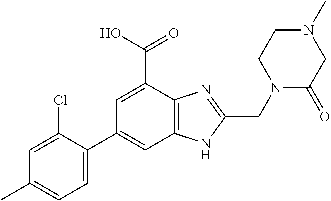 [SMILES: Cc1ccc(-c2cc(C(=O)O)c3nc(CN4CCN(C)CC4=O)[nH]c3c2)c(Cl)c1]

The title compound was obtained in comparable yield analogous to the procedure described for Example 7 using 4-methylpiperazin-2-one instead of hexahydropyrrolo[1,2-a]pyrazin-6(2H)-one in step b), light yellow solid, MS (ESI): 413.23 [M+H]+.

### Example 13

### 6-(2-Chloro-4-methylphenyl)-2-(hydroxymethyl)-1H-benzimidazole-4-carboxylic acid

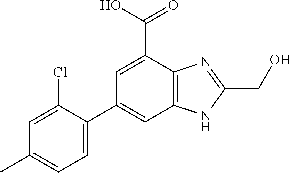 [SMILES: Cc1ccc(-c2cc(C(=O)O)c3nc(CO)[nH]c3c2)c(Cl)c1]

### a) Methyl 6-(2-chloro-4-methylphenyl)-2-(hydroxymethyl)-1H-benzimidazole-4-carboxylate

To a light yellow solution of methyl 6-bromo-2-(chloromethyl)-1H-benzo[d]imidazole-4-carboxylate (see Example 7, 40 mg, 132 μmol, Eq: 1) in dioxane (2 ml) was added (2-chloro-4-methylphenyl)boronic acid (23.6 mg, 138 μmol, Eq: 1.05). Potassium phosphate (tribasic) (55.9 mg, 264 μmol, Eq: 2) solved in water (500 μl) was added. The mixture was degassed during 2 min before X-phos (3.14 mg, 6.59 μmol, Eq: 0.05) and tris(dibenzylideneacetone)dipalladium chloroform adduct (3.41 mg, 3.29 μmol, Eq: 0.025) were added. The mixture was heated to 100° C. for 1 hours. For work-up the reaction mixture was poured into 20 ml of water and extracted with EtOAc (3×20 ml). The organic layers were dried over MgSO₄ and concentrated in vacuo. The crude material was purified by flash chromatography (silica gel, 12 g, 0% to 10% MeOH in DCM) to afford the title compound methyl 6-(2-chloro-4-methylphenyl)-2-(hydroxymethyl)-1H-benzo[d]imidazole-4-carboxylate as light yellow solid, MS (ESI): 331.09 [M+H]+.

### b) 6-(2-Chloro-4-methylphenyl)-2-(hydroxymethyl)-1H-benzimidazole-4-carboxylic acid

To a light yellow solution of methyl 6-(2-chloro-4-methylphenyl)-2-(hydroxymethyl)-1H-benzo[d]imidazole-4-carboxylate (12 mg, 36.3 Eq: 1) in tetrahydrofuran (1 ml) was added lithium hydroxide monohydrate (3.04 mg, 72.6 μmol, Eq: 2) dissolved in water (250 μL). The mixture was heated to 65° C. during 4 h, then quenched with HCl (2M, 36.3 μl, 72.6 μmol, Eq: 2) and concentrated in vacuo. The crude material was triturated with diethyl ether (2×5 ml) to obtain the title compound 6-(2-chloro-4-methylphenyl)-2-(hydroxymethyl)-1H-benzimidazole-4-carboxylic acid (12.1 mg, 33.3 μmol, 91.9% yield) as light yellow solid, MS (ESI): 317.13 [M+H]+.

### Example 14

### 6-(2-Chloro-4-methylphenyl)-2-(phenylamino)-1H-benzo[d]imidazole-4-carboxylic acid

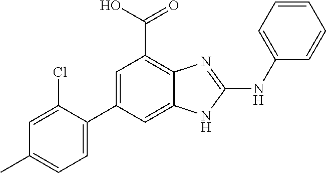 [SMILES: Cc1ccc(-c2cc(C(=O)O)c3nc(Nc4ccccc4)[nH]c3c2)c(Cl)c1]

### a) Methyl 4,5-diamino-2′-chloro-4′-methyl-[1,1′-biphenyl]-3-carboxylate

To a solution of methyl 2,3-diamino-5-bromobenzoate (2 g, 8.16 mmol, Eq: 1) in dioxane (40 ml) was added (2-chloro-4-methylphenyl)boronic acid (1.39 g, 8.16 mmol, Eq: 1). Potassium phosphate (tribasic) (3.46 g, 16.3 mmol, Eq: 2) dissolved in water (10 ml) was added. The reaction mixture was degassed during 2 min before X-phos (195 mg, 408 μmol, Eq: 0.05) and tris(dibenzylideneacetone)dipalladium chloroform adduct (211 mg, 204 μmol, Eq: 0.025) were added. The mixture was heated to 110° C. for 2 hours, then poured into 100 ml of water and extracted with EtOAc (3×100 ml). The organic layers were dried over MgSO₄ and concentrated in vacuo. The crude material was purified by flash chromatography (silica gel, 80 g, 0% to 70% EtOAc in heptane) to afford the title compound methyl 4,5-diamino-2′-chloro-4′-methyl-[1,1′-biphenyl]-3-carboxylate (2.06 g, 6.85 mmol, 84% yield) as dark brown gum, 291.11 [M+H]+.

### b) Methyl 2-((tert-butoxycarbonyl)amino)-6-(2-chloro-4-methylphenyl)-1H-benzo[d]imidazole-4-carboxylate

A mixture of methyl 4,5-diamino-2′-chloro-4′-methyl-[1,1′-biphenyl]-3-carboxylate (450 mg, 1.55 mmol, Eq: 1), 1,3-di-boc-2-methylisothiourea (714 mg, 2.38 mmol, Eq: 1.54) and (+)-camphor-10-sulfonic acid monohydrate (27.3 mg, 107 μmol, Eq: 0.069) in ethanol (10.8 ml) was refluxed for 3 h, then the mixture was cooled to room temperature and filtered. The grey filter cake was washed three times with 2 ml of ethanol and dried to afford the title compound methyl 2-((tert-butoxycarbonyl)amino)-6-(2-chloro-4-methylphenyl)-1H-benzo[d]imidazole-4-carboxylate (582 mg, 1.35 mmol, 87.3% yield) as grey solid, 416.23 [M+H]+.

### c) Methyl 2-amino-6-(2-chloro-4-methylphenyl)-1H-benzo[d]imidazole-4-carboxylate

In a 25 ml round-bottomed flask, methyl 2-((tert-butoxycarbonyl)amino)-6-(2-chloro-4-methylphenyl)-1H-benzo[d]imidazole-4-carboxylate (778 mg, 1.87 mmol, Eq: 1) and HCl (1N in dioxane, 11.7 ml, 46.8 mmol, Eq: 25) were combined with dioxane (6.68 ml). The reaction was heated for 5 h at 60° C. For work-up the reaction mixture was diluted with diethyl ether and heptan, the precipitate was filtered off, dried in vacuo to afford the title compound methyl 2-amino-6-(2-chloro-4-methylphenyl)-1H-benzo[d]imidazole-4-carboxylate (546 mg, 1.73 mmol, 92% yield) as an off-white solid, MS (ESI): 316.12 [M+H]+.

### d) Methyl 2-bromo-6-(2-chloro-4-methylphenyl)-1H-benzo[d]imidazole-4-carboxylate

To a dark green solution of tert.-butyl nitrite (196 mg, 226 μl, 1.9 mmol, Eq: 1.5) and copper (II) bromide (424 mg, 1.9 mmol, Eq: 1.5) in acetonitrile (12 ml) at 60° C. methyl 2-amino-6-(2-chloro-4-methylphenyl)-1H-benzo[d]imidazole-4-carboxylate (400 mg, 1.27 mmol, Eq: 1) was added portionwise. After complete addition the mixture was heated up to 75° C. for 2 hours. The reaction mixture was quenched with 100 ml of 1M HCl and extracted with EtOAc (3×100 ml). The organic layers were dried over MgSO₄ and concentrated in vacuo. Crude material was suspended in MeOH (10 ml). Suspension was stirred for 30 min before solids were filtered off. Solids were washed with MeOH. The crude material (487 mg) was purified by preparative HPLC (Gemini NX, 12 nm, 5 μm, 100×30 mm, eluent acetonitrile/water) to obtain the title compound methyl 2-bromo-6-(2-chloro-4-methylphenyl)-1H-benzo[d]imidazole-4-carboxylate (91.7 mg, 229 μmol, 18.1% yield) as a white solid, MS (ESI): 381.04 [M+H]+.

### e) Methyl 6-(2-chloro-4-methylphenyl)-2-(phenylamino)-1H-benzo[d]imidazole-4-carboxylate

In a 5 ml vial, methyl 2-bromo-6-(2-chloro-4-methylphenyl)-1H-benzo[d]imidazole-4-carboxylate (40 mg, 105 μmol, Eq: 1) and aniline (98.1 mg, 96.2 μl, 1.05 mmol, Eq: 10) were combined with ethanol (0.5 ml). The reaction mixture was heated to 90° C. and stirred overnight. For work-up the reaction mixture was poured into 20 ml of 1M HCl and extracted with EtOAc (3×25 ml). The organic layers were combined, dried over MgSO₄, filtered through sintered glass, concentrated and dried in vacuo. The crude material was purified by chromatography (silica gel, 12 g, 0% to 50% EtOAc in heptane to afford the title compound methyl 6-(2-chloro-4-methylphenyl)-2-(phenylamino)-1H-benzo[d]imidazole-4-carboxylate (36.4 mg, 91.5 μmol, 86.8% yield), off-white solid, MS (ESI): 392.19 [M+H]+.

### f) 6-(2-Chloro-4-methylphenyl)-2-(phenylamino)-1H-benzo[d]imidazole-4-carboxylic acid

To a light yellow solution of methyl 6-(2-chloro-4-methylphenyl)-2-(phenylamino)-1H-benzo[d]imidazole-4-carboxylate (19 mg, 48.5 μmol, Eq: 1) in tetrahydrofuran (500 μl) was added lithium hydroxide (4.07 mg, 97 μmol, Eq: 2) dissolved in water (250 μl). The reaction mixture was heated to 65° C. during 4 hours, then quenched with HCl (48.5 μl, 97 μmol, Eq: 2) and concentrated in vacuo. The crude material was dissolved with a few drops of diethylether and than triturated with Heptan (2×10 ml) to obtain the title compound 6-(2-chloro-4-methylphenyl)-2-(phenylamino)-1H-benzo[d]imidazole-4-carboxylic acid (18 mg, 47.1 μmol, 97.2% yield) as white solid, MS (ESI): 378.13 [M+H]+.

### Example 15

### 6-(2-Chloro-4-methylphenyl)-2-[(4,4-difluoropiperidin-1-yl)methyl]-1H-benzimidazole-4-carboxylic acid

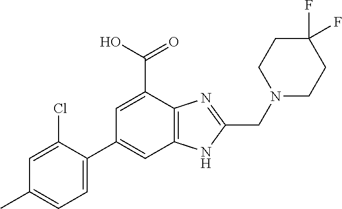 [SMILES: Cc1ccc(-c2cc(C(=O)O)c3nc(CN4CCC(F)(F)CC4)[nH]c3c2)c(Cl)c1]

The title compound was obtained in comparable yield analogous to the procedure described for Example 7 using 4,4-difluoropiperidine hydrochloride instead of hexahydropyrrolo[1,2-a]pyrazin-6(2H)-one in step b), light red solid, MS (ESI): 420.21 [M+H]+.

### Example 16

### 2-[(4-Acetylpiperazin-1-yl)methyl]-6-(2-chloro-4-methylphenyl)-1H-benzimidazole-4-carboxylic acid

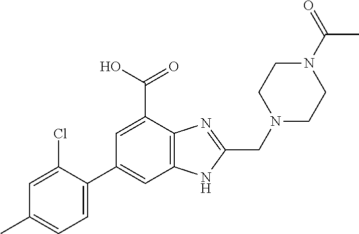 [SMILES: CC(=O)N1CCN(Cc2nc3c(C(=O)O)cc(-c4ccc(C)cc4Cl)cc3[nH]2)CC1]

The title compound was obtained in comparable yield analogous to the procedure described for Example 7 using 1-acetylpiperazine instead of hexahydropyrrolo[1,2-a]pyrazin-6(2H)-one in step b), white solid, MS (ESI): 427.26 [M+H]+.

### Example 17

### 6-(2-Chloro-4-methylphenyl)-2-[(N-methylanilino)methyl]-1H-benzimidazole-4-carboxylic acid

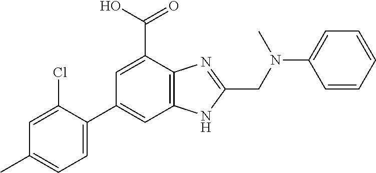 [SMILES: Cc1ccc(-c2cc(C(=O)O)c3nc(CN(C)c4ccccc4)[nH]c3c2)c(Cl)c1]

The title compound was obtained in comparable yield analogous to the procedure described for Example 7 using N-methylaniline instead of hexahydropyrrolo[1,2-a]pyrazin-6(2H)-one in step b), light yellow solid, MS (ESI): 406.18 [M+H]+.

### Example 18

### 2-[(4-Benzylpiperazin-1-yl)methyl]-6-(2-chloro-4-methylphenyl)-1H-benzimidazole-4-carboxylic acid

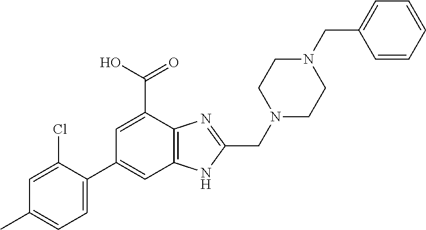 [SMILES: Cc1ccc(-c2cc(C(=O)O)c3nc(CN4CCN(Cc5ccccc5)CC4)[nH]c3c2)c(Cl)c1]

The title compound was obtained in comparable yield analogous to the procedure described for Example 7 using 1-benzyl-piperazine instead of hexahydropyrrolo[1,2-a]pyrazin-6(2H)-one in step b), light brown solid, MS (ESI): 475.28 [M+H]+.

### Example 19

### 6-(2-Chloro-4-methylphenyl)-2-[(4-phenylpiperazin-1-yl)methyl]-1H-benzimidazole-4-carboxylic acid

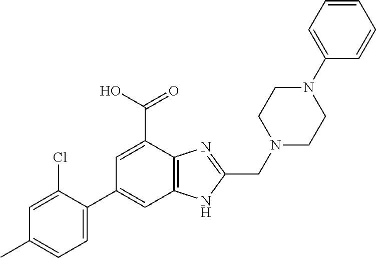 [SMILES: Cc1ccc(-c2cc(C(=O)O)c3nc(CN4CCN(c5ccccc5)CC4)[nH]c3c2)c(Cl)c1]

The title compound was obtained in comparable yield analogous to the procedure described for Example 7 using 1-phenyl-piperazine instead of hexahydropyrrolo[1,2-a]pyrazin-6(2H)-one in step b), light brown solid, MS (ESI): 461.29 [M+H]+.

### Example 20

### 6-(2-Chloro-4-methylphenyl)-2-[(3-oxopiperazin-1-yl)methyl]-1H-benzimidazole-4-carboxylic acid

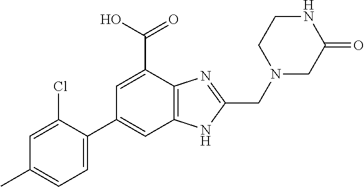 [SMILES: Cc1ccc(-c2cc(C(=O)O)c3nc(CN4CCNC(=O)C4)[nH]c3c2)c(Cl)c1]

The title compound was obtained in comparable yield analogous to the procedure described for Example 7 using piperazin-2-one instead of hexahydropyrrolo[1,2-a]pyrazin-6(2H)-one in step b), off-white solid, MS (ESI): 399.17 [M+H]+.

### Example 21

### 6-(2-Chloro-4-methylphenyl)-2-[(4-hydroxypiperidin-1-yl)methyl]-1H-benzimidazole-4-carboxylic acid

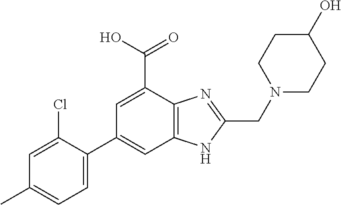 [SMILES: Cc1ccc(-c2cc(C(=O)O)c3nc(CN4CCC(O)CC4)[nH]c3c2)c(Cl)c1]

The title compound was obtained in comparable yield analogous to the procedure described for Example 7 using piperidin-4-ol instead of hexahydropyrrolo[1,2-a]pyrazin-6(2H)-one in step b), white solid, MS (ESI): 400.18 [M+H]+.

### Example 22

### 6-(2-Chloro-4-methylphenyl)-2-[(4-methylpiperazin-1-yl)methyl]-1-benzimidazole-4-carboxylic acid

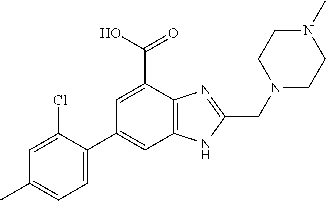

The title compound was obtained in comparable yield analogous to the procedure described for Example 7 using 1-methyl-piperazine instead of hexahydropyrrolo[1,2-a]pyrazin-6(2H)-one in step b), white solid, MS (ESI): 399.158 [M+H]+.

### Example 23

### 6-(2-Chloro-4-methylphenyl)-2-(diethylaminomethyl)-1-benzimidazole-4-carboxylic acid

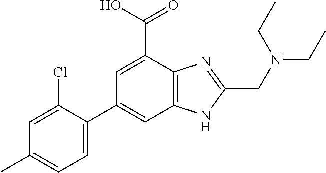 [SMILES: CCN(CC)Cc1nc2c(C(=O)O)cc(-c3ccc(C)cc3Cl)cc2[nH]1]

The title compound was obtained in comparable yield analogous to the procedure described for Example 7 using diethylamine instead of hexahydropyrrolo[1,2-a]pyrazin-6(2H)-one in step b), light brown solid, MS (ESI): 372.147 [M+H]+.

### Example 24

### 6-(2-Chloro-4-methylphenyl)-2-(piperidin-1-ylmethyl)-1H-benzimidazole-4-carboxylic acid

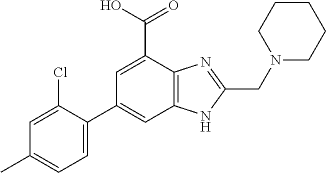 [SMILES: Cc1ccc(-c2cc(C(=O)O)c3nc(CN4CCCCC4)[nH]c3c2)c(Cl)c1]

The title compound was obtained in comparable yield analogous to the procedure described for Example 7 using piperidine instead of hexahydropyrrolo[1,2-a]pyrazin-6(2H)-one in step b), light brown solid, MS (ESI): 384.20 [M+H]+.

### Example 25

### 2-(Anilinomethyl)-6-(2-chloro-4-methylphenyl)-1H-benzimidazole-4-carboxylic acid

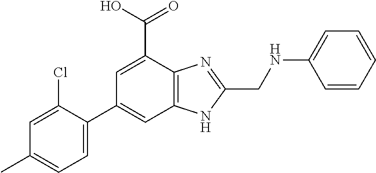 [SMILES: Cc1ccc(-c2cc(C(=O)O)c3nc(CNc4ccccc4)[nH]c3c2)c(Cl)c1]

The title compound was obtained in comparable yield analogous to the procedure described for Example 7 using analine instead of hexahydropyrrolo[1,2-a]pyrazin-6(2H)-one in step b), white solid, MS (ESI): 392.19 [M+H]+.

### Example 26

### 2-Benzyl-6-(2-chloro-4-methylphenyl)-1H-benzo[d]imidazole-4-carboxylic acid

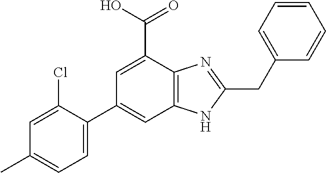 [SMILES: Cc1ccc(-c2cc(C(=O)O)c3nc(Cc4ccccc4)[nH]c3c2)c(Cl)c1]

### a) Methyl 2-benzyl-6-bromo-1H-benzimidazole-4-carboxylate

To a mix of methyl 2,3-diamino-5-bromobenzoate (100 mg, 408 μmol, Eq: 1) and 2-phenylacetic acid (55.6 mg, 408 μmol, Eq: 1) under argon was added phosphorus oxychloride (1 ml). Vial was closed and RM was heated to 110° C. for 2 hours. The reaction mixture was quenched by adding it slowly on 25 ml of saturated NaHCO₃ solution. The aqueous phase was extracted with EtOAc (3×25 ml). The organic layers were dried over MgSO₄ and concentrated in vacuo. The crude material was purified by flash chromatography (silica gel, 12 g, 0% to 70% EtOAc in heptane) to afford the title compound methyl 2-benzyl-6-bromo-1H-benzo[d]imidazole-4-carboxylate (113 mg, 315 μmol, 77.3% yield) as off-white solid, MS (ESI): 347.07 [M+H]+.

### b) Methyl 2-benzyl-6-(2-chloro-4-methylphenyl)-1H-benzo[d]imidazole-4-carboxylate

Methyl 2-benzyl-6-bromo-1H-benzo[d]imidazole-4-carboxylate (107 mg, 310 μmol, Eq: 1), (2-chloro-4-methylphenyl)boronic acid (52.8 mg, 310 μmol, Eq: 1) and potassium carbonate (tribasic) (132 mg, 620 μmol, Eq: 2) were combined with dioxane (1.71 ml) and water (428 μl). The vial was degassed with argon before X-phos (7.39 mg, 15.5 μmol, Eq: 0.05) and tris(dibenzylideneacetone)dipalladium chloroform adduct (8.02 mg, 7.75 μmol, Eq: 0.025) were added. The vial was closed and the reaction mixture was heated to 110° C. and stirred for 1.5 h. For work-up the reaction mixture was poured into 20 ml of water and extracted with EtOAc (3×20 ml). The organic layers were combined, dried over Na₂SO₄, filtered through sintered glass, concentrated and dried in vacuo. The crude material was purified by flash chromatography (silica gel, 12 g, 0% to 60% EtOAc in heptane) to afford the title compound methyl 2-benzyl-6-(2-chloro-4-methylphenyl)-1H-benzo[d]imidazole-4-carboxylate (68.2 mg, 167 μmol, 53.9% yield) as brown oil, MS (ESI): 391.121 [M+H]+.

### c) 2-Benzyl-6-(2-chloro-4-methylphenyl)-1H-benzo[d]imidazole-4-carboxylic acid

To a colorless solution of methyl 2-benzyl-6-(2-chloro-4-methylphenyl)-1H-benzo[d]imidazole-4-carboxylate (63.2 mg, 162 μmol, Eq: 1) in tetrahydrofuran (2.34 ml) was added lithium hydroxide monohydrate (13.6 mg, 323 μmol, Eq: 2) dissolved in water (1.17 ml). The mixture was warmed to 65° C. and stirred for 3 h. For work-up HCl (2M, 162 μl, 323 μmol, Eq: 2) was added and concentrated in vacuo. The crude material was purified by preparative HPLC (Gemini NX, 12 nm, 5 μm, 100×30 mm, eluent acetonitrile/water) to obtain the title compound 2-benzyl-6-(2-chloro-4-methylphenyl)-1H-benzo[d]imidazole-4-carboxylic acid (3.9 mg, 9.94 μmol, 6.14% yield) as white solid, MS (ESI): 377.105 [M+H]+.

### Example 27

### 6-(2-Chloro-4-methylphenyl)-2-[(4-chlorophenyl)methyl]-1H-benzimidazole-4-carboxylic acid

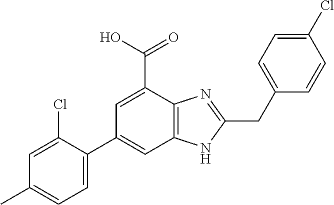 [SMILES: Cc1ccc(-c2cc(C(=O)O)c3nc(Cc4ccc(Cl)cc4)[nH]c3c2)c(Cl)c1]

The title compound was obtained in comparable yield analogous to the procedure described for Example 26 using 2-(4-chlorophenyl)acetic acid instead of 2-phenylacetic acid in step a), white solid, MS (ESI): 411.13 [M+H]+.

### Example 28

### 6-(2-Chloro-4-methylphenyl)-2-(morpholin-4-ylmethyl)-1H-benzimidazole-4-carboxylic acid

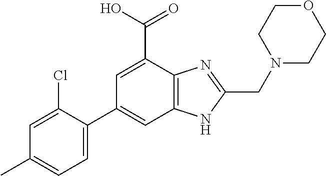 [SMILES: Cc1ccc(-c2cc(C(=O)O)c3nc(CN4CCOCC4)[nH]c3c2)c(Cl)c1]

The title compound was obtained in comparable yield analogous to the procedure described for Example 7 using morpholine instead of hexahydropyrrolo[1,2-a]pyrazin-6(2H)-one in step b), light brown solid, MS (ESI): 386.19 [M+H]+.

### Example 29

### 6-(2-Chloro-4-methylphenyl)-2-[(dimethylamino)methyl]-1H-benzimidazole-4-carboxylic acid

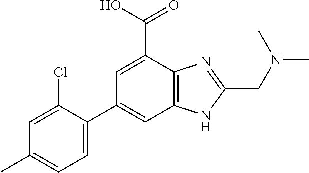

The title compound was obtained in comparable yield analogous to the procedure described for Example 7 using dimethyl amine instead of hexahydropyrrolo[1,2-a]pyrazin-6(2H)-one in step b), white solid, MS (ESI): 344.14 [M+H]+.

### Example 30

### 6-(2-Chloro-4-methylphenyl)-2-(trifluoromethyl)-1H-benzimidazole-4-carboxylic acid

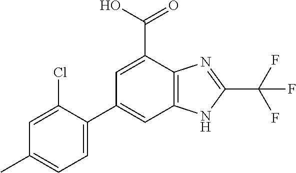 [SMILES: Cc1ccc(-c2cc(C(=O)O)c3nc(C(F)(F)F)[nH]c3c2)c(Cl)c1]

### a) Methyl 6-bromo-2-(trifluoromethyl)-1H-benzimidazole-4-carboxylate

Methyl 2,3-diamino-5-bromobenzoate (100 mg, 408 Eq: 1) was combined with TFA (1.5 ml) to give a light yellow solution. Vial was closed under Argon and heated to 70° C. over night. LC-MS showed the reaction was complete. The reaction mixture was quenched with 25 ml sat NaHCO₃ and extracted with DCM (3×25 ml). The organic layers were dried over MgSO₄ and concentrated in vacuo to obtain the crude title compound methyl 6-bromo-2-(trifluoromethyl)-1H-benzo[d]imidazole-4-carboxylate (124 mg, 376 μmol, 92.3% yield) as light brown solid, MS (ESI): 323.00 [M+H]+.

### b) 6-(2-Chloro-4-methylphenyl)-2-(trifluoromethyl)-1H-benzimidazole-4-carboxylic acid

The title compound was obtained in comparable yield analogous to the procedure described for Example 26 using methyl 6-bromo-2-(trifluoromethyl)-1H-benzimidazole-4-carboxylate instead of methyl 2-benzyl-6-bromo-1H-benzo[d]imidazole-4-carboxylate in step b), white solid, MS (ESI): 355.09 [M+H]+.

### Example 31

### 2-(Benzylamino)-6-(2-chloro-4-methylphenyl)-1H-benzo[d]imidazole-4-carboxylic acid

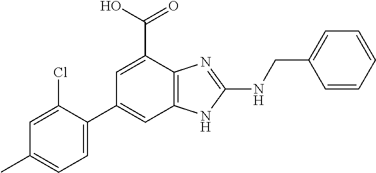 [SMILES: Cc1ccc(-c2cc(C(=O)O)c3nc(NCc4ccccc4)[nH]c3c2)c(Cl)c1]

### a) Methyl 6-bromo-2-((tert-butoxycarbonyl)amino)-1H-benzo[d]imidazole-4-carboxylate

A mixture of methyl 2,3-diamino-5-bromobenzoate (200 mg, 816 μmol, Eq: 1), 1,3-di-boc-2-methylisothiourea (376 mg, 1.26 mmol, Eq: 1.54) and (+)-camphor-10-sulfonic acid monohydrate (14.4 mg, 56.3 μmol, Eq: 0.069) in ethanol (6 ml) was refluxed for 3 h. For work-up the reaction mixture was cooled to room temperature and filtered. The grey filter cake was washed three times with EtOH and dried. It was used without purification in the next step, (281.5 mg, 760 μmol, 93.1% yield) as grey solid, MS (ESI): 370.0372 [M+H]+.

### b) Methyl 2-(benzyl(tert-butoxycarbonyl)amino)-6-bromo-1H-benzo[d]imidazole-4-carboxylate

In a three-necked flask, methyl 6-bromo-2-((tert-butoxycarbonyl)amino)-1H-benzo[d]imidazole-4-carboxylate (150 mg, 405 μmol, Eq: 1) and cesium carbonate (158 mg, 486 μmol, Eq: 1.2) were combined with dimethylformamide (12 ml) to give a grey suspension. Then benzylbromide (84.9 mg, 59 μl, 486 μmol, Eq: 1.2) was added at room temperature. The reaction mixture was stirred at room temperature overnight. For work-up the mixture was concentrated in vacuo, the residue was diluted with dichloromethane and evaporated with silica gel to dryness and separated from regioisomers by flash chromatography (silica gel, 80 g, 0% to 40% EtOAc in heptane) to afford the title compound methyl 2-(benzyl(tert-butoxycarbonyl)amino)-6-bromo-1H-benzo[d]imidazole-4-carboxylate (108 mg, 235 μmol, 58.0% yield) as white foam, MS (ESI): 458.1, 460.1 [M−H]−.

### c) Methyl 2-(benzyl(tert-butoxycarbonyl)amino)-6-(2-chloro-4-methylphenyl)-1H-benzo[d]imidazole-4-carboxylate

Methyl 2-(benzyl(tert-butoxycarbonyl)amino)-6-bromo-1H-benzo[d]imidazole-4-carboxylate (50 mg, 109 Eq: 1) and (2-chloro-4-methylphenyl)boronic acid (27.8 mg, 163 μmol, Eq: 1.5) were solved in 1,4-dioxane (3 ml) and water (1.5 ml). Cesium carbonate (143 mg, 434 μmol, Eq: 4) was added and the mixture was degassed by bubbling argon through the mixture (5 min), then 1,1′-bis(diphenylphosphino)ferrocene-palladium(II)dichloride dichloromethane complex (4.44 mg, 5.43 μmol, Eq: 0.05) was added. The reaction was stirred in a sealed tube at 90° C. for 15 min. For work-up the mixture was taken up in EtOAc and washed with saturated NH₄Cl solution and brine, the organic layer was dried over Na₂SO₄, filtrated and evaporated. The residue was purified by flash chromatography (silica gel, 40 g, 0% to 20% EtOAc in heptane) to afford the title compound methyl 2-(benzylamino)-6-(2-chloro-4-methylphenyl)-1H-benzo[d]imidazole-4-carboxylate (51.5 mg, 102 μmol, 93.5% yield) as white foam, MS (ESI): 506.2 [M+H]+.

### d) Methyl 2-(benzylamino)-6-(2-chloro-4-methylphenyl)-1H-benzo[d]imidazole-4-carboxylate

Methyl 2-(benzyl(tert-butoxycarbonyl)amino)-6-(2-chloro-4-methylphenyl)-1H-benzo[d]imidazole-4-carboxylate (94 mg, 182 μmol, Eq: 1) was solved in 1,4-dioxane (1 ml). At room temperature 4M HCl in dioxane (1.39 ml, 5.56 mmol, Eq: 30.5) was added. The reaction mixture was stirred overnight at room temperature for 2 days. For work-up the mixture was taken up in EtOAc and washed with saturated NaHCO₃ solution. The organic layer was dried over Na₂SO₄, filtrated and evaporated. The residue was purified by flash chromatography (silica gel, 40 g, 0% to 65% EtOAc in heptane) to afford the title compound methyl 2-(benzylamino)-6-(2-chloro-4-methylphenyl)-1H-benzo[d]imidazole-4-carboxylate (51.3 mg, 126 μmol, 69.2% yield) as white foam, MS (ESI): 406.2 [M+H]+.

### e) 2-(Benzylamino)-6-(2-chloro-4-methylphenyl)-1H-benzo[d]imidazole-4-carboxylic acid

Methyl 2-(benzylamino)-6-(2-chloro-4-methylphenyl)-1H-benzo[d]imidazole-4-carboxylate (49.5 mg, 120 μmol, Eq: 1) was solved in THF (1 ml). At room temperature aqueous lithium hydroxide solution (1M, 301 μl, 301 μmol, Eq: 2.5) was added and the mixture was stirred at 65° C. overnight. For work-up the mixture was diluted with water, HCl (2M, 150 μl, 301 μmol, Eq: 2.5) was added and the pH was adjusted to 3. The mixture was extracted three times with 2-methyltetrahydrofuran, the combined organic layers were dried over Na₂SO₄, filtrated and evaporated to afford the title compound 2-(benzylamino)-6-(2-chloro-4-methylphenyl)-1H-benzo[d]imidazole-4-carboxylic acid (44 mg, 112 μmol, 93.3% yield) as white solid, MS (ESI): 392.3 [M+H]+.

### Example 32

### 6-(2-Chloro-4-methylphenyl)-1H-benzo[d]imidazole-4-carboxylic acid

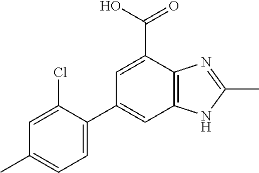

The title compound was obtained in comparable yield analogous to the procedure described for Example 26 using methyl 6-bromo-2-methyl-1H-benzimidazole-4-carboxylate instead of methyl 2-benzyl-6-bromo-1H-benzo[d]imidazole-4-carboxylate in step b), white solid, MS (ESI): 301.1 [M+H]+.

### Example 33

Malachite Green Assay to Measure cGAS Activity
Compounds were tested for cGAS inhibition in a coupled enzymatic assay based on Phosphate detection by Malachite Green. Final assay conditions were 20 mM TRIS pH 7.5 (Applichem), 5 mM MgCl₂ (Sigma) and 0.01% BSA (Sigma) supplemented with 80 ATP (Sigma), 80 μM GTP (Sigma) and 100 nM Interferon Stimulating DNA (ISD) (Microsynth). Recombinantly expressed purified human cGAS (residues 161-522) was used at 25 nM.
All compounds were prepared as 10 mM stock solutions in DMSO and a 16 pt dilution series in DMSO with a dilution factor of 2.5 was prepared. 1 μL of DMSO dilution series was transferred to 32.3 μL reaction buffer, mixed by pipetting up/down, spun for 1 minute at 3000 rpm and was visually inspected for precipitation. 5 μL of 3-fold enzyme stock solution were transferred to an empty 384-well Black/Clear Flat Bottom Polystyrene NBS (Corning) rows 3-24. Rows 1-2 were filled with assay buffer. Plates were spun 10 seconds at 1000 rpm (164×g). 5 μL of compound intermediate dilution was added and mixed by pipetting up/down to rows 3-24. Rows 1-2 were filled with 3.1% DMSO assay buffer. Plates were spun 10 seconds at 1000 rpm (164×g). 5 μL 3-fold Nucleotide/DNA mix was added to all wells to start the reaction. Plates were spun 10 seconds at 1000 rpm (164×g) and incubated for 4 hour at room temperature (RT) in the dark. 5 μL 4 U/mL PPase (Sigma) were added to all wells. Plates spun 10 seconds at 1000 rpm (164×g). 10 μL BioMol green Solution (Enzo Life Sciences) was added to all wells. Plates spun 10 seconds at 1000 rpm (164×g) and incubated 30 minutes at RT in the dark. Absorbance data was collected 620 nm on an EnVision Multilable Reader (Perkin Elmer) and the following measurement settings were used: excitation filter photometric was 620 nm; excitation from the top; measurement height was 1 mm; number of flashes was 30; number of flashes integrated was 1.
All plates are checked for abnormalities and outliers in the Blank Control (no protein, row 1) and the Neutral Control (no compound, row 2) are excluded using the 3*SD rule. Data was normalized to 0 and 100% by Blank and Neutral Control and each curve was fitted and judged using the 4 parameter logistic equation to determine the IC50 for cGAS inhibition.
The results of this assay are provided in Table 1. Table 1 provides IC50 values (μM) for cGAS inhibition obtained for particular examples of the present invention as measured by the above-described assay.

| Example | IC50 cGAS (μM) |
|---|---|
| 1 | 4.68 |
| 2 | 0.54 |
| 3 | 0.55 |
| 4 | 4.37 |
| 5 | 3.26 |
| 6 | 2.08 |
| 7 | 0.26 |
| 8 | 0.59 |
| 9 | 0.38 |
| 10 | 0.51 |
| 11 | 0.60 |
| 12 | 1.14 |
| 13 | 0.55 |
| 14 | 1.16 |
| 15 | 0.26 |
| 16 | 1.51 |
| 17 | 1.23 |
| 18 | 0.14 |
| 19 | 1.25 |
| 20 | 0.59 |
| 21 | 0.41 |
| 22 | 0.13 |
| 23 | 0.48 |
| 24 | 0.36 |
| 25 | 0.69 |
| 26 | 1.02 |
| 27 | 1.48 |
| 28 | 0.35 |
| 29 | 0.59 |
| 30 | 3.02 |
| 31 | 0.84 |
| 32 | 2.41 |

### Example A

Film coated tablets containing the following ingredients can be manufactured in a conventional manner:

| | |
|---|---|
| Ingredients | Per tablet |

Kernel:

| | | | | |
|---|---|---|---|---|
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule | |
|---|---|---|
| Compound of formula (I) | 25.0 | mg |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| | | |
|---|---|---|
| Compound of formula (I) | 3.0 | mg |
| Polyethylene glycol 400 | 150.0 | mg |

| | |
|---|---|
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 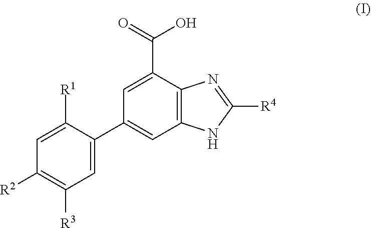
wherein
R¹ is alkyl or halogen;
R² is alkyl, halogen, haloalkyl, alkoxy or cycloalkyl;
R³ is hydrogen or halogen; and
R⁴ is hydrogen, (oxo-hexahydropyrrolo[1,2-a]pyrazinyl)alkyl, haloalkylpiperazinylalkyl, cycloalkylpiperazinylalkyl, phenylalkyl(oxopiperazinyl)alkyl, alkyl(oxopiperazinyl)alkyl, hydroxyalkyl, phenylamino, halopiperidinylalkyl, alkylcarbonylpiperazinylalkyl, phenyl(alkylamino)alkyl, phenylalkylpiperazinylalkyl, phenylpiperazinylalkyl, oxopiperazinylalkyl, hydroxypiperidinylalkyl, alkylpiperazinylalkyl, dialkylaminoalkyl, piperidinylalkyl, phenylaminoalkyl, phenylalkyl, halophenylalkyl, morpholinylalkyl, haloalkyl, alkyl or phenylalkylamino;
or a pharmaceutically acceptable salt or ester thereof.

2. A compound according to claim 1, wherein R¹ is halogen.

3. A compound according to claim 1 or 2, wherein R¹ is chlorine.

4. A compound according to any one of claims 1 to 3, wherein R² is alkyl.

5. A compound according to any one of claims 1 to 4, wherein R² is methyl.

6. A compound according to any one of claims 1 to 5, wherein R³ is hydrogen or fluorine.

7. A compound according to any one of claims 1 to 6, wherein R⁴ is hydrogen, (oxo-hexahydropyrrolo[1,2-a]pyrazinyl)alkyl, cycloalkylpiperazinylalkyl, hydroxyalkyl, halopiperidinylalkyl, phenylalkylpiperazinylalkyl, oxopiperazinylalkyl, alkylpiperazinylalkyl, piperidinylalkyl, phenylaminoalkyl, phenylalkyl, morpholinylalkyl or phenylalkylamino.

8. A compound according to any one of claims 1 to 7, wherein R⁴ is hydrogen, (oxo-hexahydropyrrolo[1,2-a]pyrazinyl)methyl, cyclopropylpiperazinylmethyl, hydroxymethyl, halopiperidinylmethyl, phenylmethylpiperazinylmethyl, oxopiperazinylmethyl, methylpiperazinylmethyl, piperidinylmethyl, phenylaminomethyl, phenylmethyl, morpholinylmethyl or phenylmethylamino.

9. A compound according to any one of claims 1 to 8 selected from
6-(2-chloro-4-cyclopropylphenyl)-1-benzimidazole-4-carboxylic acid;
6-(2-chloro-5-fluoro-4-methylphenyl)-1H-benzo[d]imidazole-4-carboxylic acid;
6-(2-chloro-4-methylphenyl)-1H-benzo[d]imidazole-4-carboxylic acid;
6-(2-chloro-4-ethoxyphenyl)-1H-benzo[d]imidazole-4-carboxylic acid;
6-(2-chloro-4-(trifluoromethyl)phenyl)-1H-benzo[d]imidazole-4-carboxylic acid;
6-(4-chloro-2-methylphenyl)-1H-benzo[d]imidazole-4-carboxylic acid;
6-(2-chloro-4-methylphenyl)-2-[(6-oxo-1,3,4,7,8,8a-hexahydropyrrolo[1,2-a]pyrazin-2-yl)methyl]-1H-benzimidazole-4-carboxylic acid;
6-(2-chloro-4-methylphenyl)-2-[[4-(2,2,2-trifluoroethyl)piperazin-1-yl]methyl]-1H-benzimidazole-4-carboxylic acid;
6-(2-chloro-4-methylphenyl)-2-[(4-cyclopropylpiperazin-1-yl)methyl]-1H-benzimidazole-4-carboxylic acid;
2-[(4-benzyl-3-oxopiperazin-1-yl)methyl]-6-(2-chloro-4-methylphenyl)-1H-benzimidazole-4-carboxylic acid;
6-(2-chloro-4-methylphenyl)-2-[[3-(trifluoromethyl)piperazin-1-yl]methyl]-1H-benzimidazole-4-carboxylic acid;
6-(2-chloro-4-methylphenyl)-2-[(4-methyl-2-oxopiperazin-1-yl)methyl]-1H-benzimidazole-4-carboxylic acid;
6-(2-chloro-4-methylphenyl)-2-(hydroxymethyl)-1H-benzimidazole-4-carboxylic acid;
6-(2-chloro-4-methylphenyl)-2-(phenylamino)-1H-benzo[d]imidazole-4-carboxylic acid;
6-(2-chloro-4-methylphenyl)-2-[(4,4-difluoropiperidin-1-yl)methyl]-1H-benzimidazole-4-carboxylic acid;
2-[(4-acetylpiperazin-1-yl)methyl]-6-(2-chloro-4-methylphenyl)-1H-benzimidazole-4-carboxylic acid;
6-(2-chloro-4-methylphenyl)-2-[(N-methylanilino)methyl]-1H-benzimidazole-4-carboxylic acid;
2-[(4-benzylpiperazin-1-yl)methyl]-6-(2-chloro-4-methylphenyl)-1H-benzimidazole-4-carboxylic acid;
6-(2-chloro-4-methylphenyl)-2-[(4-phenylpiperazin-1-yl)methyl]-1H-benzimidazole-4-carboxylic acid;
6-(2-chloro-4-methylphenyl)-2-[(3-oxopiperazin-1-yl)methyl]-1H-benzimidazole-4-carboxylic acid;
6-(2-chloro-4-methylphenyl)-2-[(4-hydroxypiperidin-1-yl)methyl]-1H-benzimidazole-4-carboxylic acid;
6-(2-chloro-4-methylphenyl)-2-[(4-methylpiperazin-1-yl)methyl]-1-benzimidazole-4-carboxylic acid;
6-(2-chloro-4-methylphenyl)-2-(diethylaminomethyl)-1-benzimidazole-4-carboxylic acid;
6-(2-chloro-4-methylphenyl)-2-(piperidin-1-ylmethyl)-1H-benzimidazole-4-carboxylic acid;
2-(anilinomethyl)-6-(2-chloro-4-methylphenyl)-1H-benzimidazole-4-carboxylic acid;
2-benzyl-6-(2-chloro-4-methylphenyl)-1H-benzo[d]imidazole-4-carboxylic acid;
6-(2-chloro-4-methylphenyl)-2-[(4-chlorophenyl)methyl]-1H-benzimidazole-4-carboxylic acid;
6-(2-chloro-4-methylphenyl)-2-(morpholin-4-ylmethyl)-1H-benzimidazole-4-carboxylic acid;
6-(2-chloro-4-methylphenyl)-2-[(dimethylamino)methyl]-1H-benzimidazole-4-carboxylic acid;
6-(2-chloro-4-methylphenyl)-2-(trifluoromethyl)-1H-benzimidazole-4-carboxylic acid;
2-(benzylamino)-6-(2-chloro-4-methylphenyl)-1H-benzo[d]imidazole-4-carboxylic acid; and
6-(2-chloro-4-methylphenyl)-1H-benzo[d]imidazole-4-carboxylic acid;
or a pharmaceutically acceptable salt or ester thereof.

10. A compound according to any one of claims 1 to 9 selected from
6-(2-chloro-5-fluoro-4-methylphenyl)-1H-benzo[d]imidazole-4-carboxylic acid;
6-(2-chloro-4-methylphenyl)-1H-benzo[d]imidazole-4-carboxylic acid;
6-(2-chloro-4-methylphenyl)-2-[(6-oxo-1,3,4,7,8,8a-hexahydropyrrolo[1,2-a]pyrazin-2-yl)methyl]-1H-benzimidazole-4-carboxylic acid;
6-(2-chloro-4-methylphenyl)-2-[(4-cyclopropylpiperazin-1-yl)methyl]-1H-benzimidazole-4-carboxylic acid;
6-(2-chloro-4-methylphenyl)-2-(hydroxymethyl)-1H-benzimidazole-4-carboxylic acid;
6-(2-chloro-4-methylphenyl)-2-[(4,4-difluoropiperidin-1-yl)methyl]-1H-benzimidazole-4-carboxylic acid;
2-[(4-benzylpiperazin-1-yl)methyl]-6-(2-chloro-4-methylphenyl)-1H-benzimidazole-4-carboxylic acid;
6-(2-chloro-4-methylphenyl)-2-[(3-oxopiperazin-1-yl)methyl]-1H-benzimidazole-4-carboxylic acid;
6-(2-chloro-4-methylphenyl)-2-[(4-methylpiperazin-1-yl)methyl]-1-benzimidazole-4-carboxylic acid;
6-(2-chloro-4-methylphenyl)-2-(piperidin-1-ylmethyl)-1H-benzimidazole-4-carboxylic acid;
2-(anilinomethyl)-6-(2-chloro-4-methylphenyl)-1H-benzimidazole-4-carboxylic acid;
2-benzyl-6-(2-chloro-4-methylphenyl)-1H-benzo[d]imidazole-4-carboxylic acid;
6-(2-chloro-4-methylphenyl)-2-(morpholin-4-ylmethyl)-1H-benzimidazole-4-carboxylic acid; and
2-(benzylamino)-6-(2-chloro-4-methylphenyl)-1H-benzo[d]imidazole-4-carboxylic acid;
or a pharmaceutically acceptable salt or ester thereof.

11. A process for the preparation of a compound according to any one of claims 1 to 10, comprising the saponification of compound (A1) 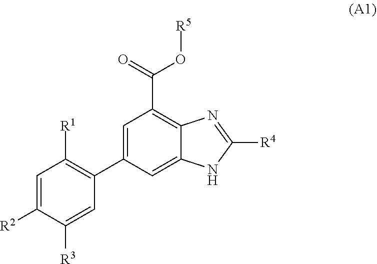
in a suitable solvent in the presence of a base or an acid;
wherein R¹, R², R³ and R⁴ are as defined in any one of claims 1 to 10 and R⁵ is alkyl.

12. A compound according to any one of claims 1 to 10, when manufactured according to a process of claim 11.

13. A compound according to any one of claims 1 to 10 for use as therapeutically active sub stance.

14. A pharmaceutical composition comprising a compound in accordance with any one of claims 1 to 10 and a therapeutically inert carrier.

15. The use of a compound according to any one of claims 1 to 10 for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

16. The use of a compound according to any one of claims 1 to 10 for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

17. A compound according to any one of claims 1 to 10 for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

18. A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound as defined in any one of claims 1 to 10 to a patient in need thereof.

19. The invention as hereinbefore described.
